(19) United States
(12) Patent Application Publication
(10) Pub. No.: US 2024/0327456 A1
(43) Pub. Date: Oct. 3, 2024

(54) CANCER THERAPEUTICS

(71) Applicant: UNIVERSITAT DE BARCELONA, Barcelona (ES)

(72) Inventors: Nieves AGELL JANE, Barcelona (ES); Montserrat JAUMOT PIJOAN, Barcelona (ES); Debora CABOT ROMERO, Barcelona (ES); Sonia BRUN LOZANO, Barcelona (ES); Baraa ABUASAKER, Barcelona (ES); Francesc RABANAL ANGLADA, Barcelona (ES); Chiara PALLARA, Barcelona (ES); Jesus SECO MORAL, Barcelona (ES); Josep RIVAS SANTOS, Barcelona (ES); Roger PRADES COSANO, Barcelona (ES); Teresa TARRAGO CLUA, Barcelona (ES)

(73) Assignee: UNIVERSITAT DE BARCELONA, Barcelona (ES)

(21) Appl. No.: 18/577,638

(22) PCT Filed: Jul. 6, 2022
(86) PCT No.: PCT/EP2022/068815

§ 371 Date: Jan. 8, 2024

(30) Foreign Application Priority Data
Jul. 7, 2021 (EP) 21382612.6
Feb. 17, 2022 (EP) 22157230.8
Feb. 18, 2022 (EP) 22157531.9

(51) Int. Cl. C07K 5/02; A61K 38/07; A61P 35/00

(52) U.S. Cl. CPC C07K 5/0202; A61K 38/07; A61P 35/00

(57) ABSTRACT

Therapeutic compounds of the invention comprise peptidomimetics, including those of formula (I), which has been prepared using solid-phase peptide synthesis. It is useful in the treatment of a cancer, including, for the treatment of a pancreatic cancer, a lung cancer or a colorectal cancer, further, including, a treatment for a pancreatic ductal adenocarcinoma.

### RELATED APPLICATIONS

This application claims priority to European patent application serial number 21382612.6 filed on Jul. 7, 2021. The contents of the aforementioned applications are incorporated herein by reference.

### FIELD OF INVENTION

This invention relates generally to cancer therapies, and more specifically, to novel therapeutic compounds comprising a peptidomimetic thereof for the treatment of a cancer.

### BACKGROUND

Cancer is generally defined as a group of diseases involving abnormal cell growth with the potential to invade or spread to other parts of the body. Cancer has been linked to several factors including smoking, obesity, poor diet, lack of physical activity and excessive consumption of alcohol. Other factors include certain infections, exposure to ionizing radiation and environmental pollutants. Certain cancers have been linked to infections such as Helicobacter pylori, hepatitis B, hepatitis C, human papillomavirus infection, Epstein-Barr virus and human immunodeficiency virus (HIV).

Conventional cancer treatments are directed at removing cancerous tissue and preventing it from spreading. Such treatment options include surgery, chemotherapy, radiation therapy, hormonal therapy, targeted therapy and palliative care. Treatments are usually pursued based on the type, location and grade of the cancer as well as the patient's health and preferences. Because cancer cells divide faster than most normal cells, they can be sensitive to chemotherapy drugs.

RAS genes comprise a family of oncogenes (HRAS, NRAS and KRAS) that are associated with cellular proliferation processes. Highly mutated forms of these RAS genes have been found in several cancers, with mutated forms of KRAS found in about 86% of RAS associated cancers and N-RAS found in about 11% and finally, HRAS found in about 3%. It is common to find a mutated RAS gene associated with some of the most deadly cancer. This includes about 90% of pancreatic cancers, 45% colon cancers and 25% of lung cancers.

The modulation of protein-protein interactions (PPIs) has gained much attention in the scientific community in recent years due to the large number of PPIs involved in the cellular machinery. However, the modulation of PPIs is highly challenging because of the nature of these interactions. An alternative to the use of small molecules for the modulation of PPIs is the use of biologics, such as antibodies. Those, which are considered large structures rather than small molecules, have the capacity to recognize and interact with large protein surfaces, but do not have the capacity to cross biological barriers, this feature limiting their therapeutic use. Thus, one aim of the present invention is to provide a PPI that is capable of recognizing and interacting with large protein surfaces while having the capacity to cross biological barriers.

As there is a need to treat cancers, including those with an association to mutated RAS genes, the present invention provides compositions and methods of treating an ailment such as cancer using therapeutics, pharmaceutical compositions thereof, and articles of manufacture.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

In an aspect of the present invention, therapeutic compounds are provided comprising a peptidomimetic that can be administered to a patient. In another aspect of the present invention, a therapeutic compound can be administered either on its own or in combination with one or more other therapeutic compounds. These additional therapeutic compounds can include an antibody, a biologic, a small molecule or other therapeutic compound as set forth in FIG. 8.

In an aspect of the present invention, a therapeutic compound that is a peptidomimetic is administered to a patient with 1, 2, 3, 4, 5, 6, 7, 8, 9, 10 or more different therapeutic compounds, including those set forth in FIG. 8.

In an aspect of the present invention, the therapeutic is a peptidomimetic, which is a small protein-like chain designed to mimic a peptide. In another aspect of the present invention, the peptidomimetic is properly engineered to recognize and bind, in a specific manner, protein patches (binding sites or sites the protein interacts with another protein or molecule, including to trigger an enzymatic pathway) and to cross biological barriers. In one aspect of the present invention, a therapeutic compound of the present invention has a molecular formula of C₄₇H₅₈N₆O₅. It can be named: (S)-N-(3-(((S)-3-amino-1-((S)-4-methyl-1-oxo-1-(pyrrolidin-1-yl) pentan-2-ylamino)-1-oxopropan-2-yl)(methyl)amino)-3-oxopropyl)-3-(biphenyl-4-yl)-2-(2,2-diphenylacetamido)-N-methylpropanamide.

In an aspect of the present invention, a therapeutic compound (I) has the capacity to efficiently inhibit the interaction of RAS (acronym from RAts Sarcoma) with its effectors (other the proteins in the cascade triggered by RAS) in cells in vitro, and it shows a high selectivity in reducing viability for cancer cells, including pancreatic tumor cells expressing an oncogenic form of KRAS. In another aspect of the present invention, a therapeutic compound inhibits the survival of PDAC cells lines, while not being toxic for non-cancerous normal cell lines. In an aspect of the present invention, a therapeutic compound and therapeutically acceptable salts thereof, are useful in the treatment of a cancer; wherein cancer selected from a pancreatic cancer, a lung cancer or a colorectal cancer; wherein the pancreatic cancer is a PDAC.

In an aspect of the present invention, a therapeutic strategy comprises a search for anti-cancer drugs that inhibit the binding of RAS with its effectors.

Another aspect of the present invention relates to pharmaceutical compositions (e.g. medicines or drugs) comprising a therapeutic compound, and a pharmaceutically acceptable salt thereof, together with pharmaceutically acceptable excipients, diluents or carriers.

Another aspect of the present invention relates to a therapeutic compound and a pharmaceutically acceptable salt thereof, for use in the treatment of a cancer, including a human cancer. This aspect may refer to the use of a therapeutic compound and a pharmaceutically acceptable salt thereof, in the manufacture of a medicine for the treatment of a cancer, including a human cancer. Alternatively, this aspect may refer to a method of treating a cancer, comprising the administration of a therapeutically effective amount of the therapeutic compound and a pharmaceutically acceptable salt thereof.

In an embodiment of the aspects mentioned in the previous paragraph, a cancer, including a human cancer is a pancreatic cancer, a lung cancer or a colorectal cancer. In other embodiments, a cancer, including a human cancer is pancreatic cancer. And, in other embodiments, a pancreatic cancer is pancreatic ductal adenocarcinoma (PDAC).

Throughout the description and claims the word “comprise” and variations of the word, are not intended to exclude other technical features, elements, limitations, additives, or components. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention.

### BRIEF DESCRIPTION OF THE DRAWINGS

The accompanying drawings illustrate aspects of the present invention. In such drawings:

FIG. 1 shows the results of co-immunoprecipitation of HA-KRASG12V with C-RAF or with PI3K in starved Hela cells expressing HA-KRASG12V after being incubated with 100 UM of compound of formula (I) for 2 h, and EGF-stimulated for 10 min (EGF=epidermal growth factor). Immunoprecipitation was performed with anti-HA antibodies and Western blot of the bound (BO) and input (IN) fractions with anti-p110αPI3K and anti-C-RAF. Experiments were repeated at least three times.

FIG. 2 shows the effect of compound of formula (I) on the cell viability (CV) of six pancreatic adenocarcinoma human cell lines (all harboring oncogenic KRAS mutations) and of a non-transformed cell line (hTERT-RPE, shown as RPE in the FIG. 2). Cells cultured in 10% FCS were treated with compound (I) in a dose range from 0 to 25 UM and incubated for 24 h, when cell viability was determined by MTS assay. Experiment was repeated 3 times. Differences were assessed using one-way ANOVA and Tukey Multiple Comparisons Test and considered significant when p≤0.05.

FIG. 3 shows the activation of RAS into a RAS GTP-bound conformation by guanine nucleotide exchange factors (GEFs) proteins state which allows RAS to associate with its protein effectors and start the downstream protein cascade. Moreover, activated RAS can be recruited by the Farnesyl transferase enzymes (FTs) and traffic through the cytosol until it reaches the cell membrane.

FIG. 4 shows a GPTase-RAS protein pharmacophoric sites (PDB: 5P21): (A) shows the complex GTPase-RAS highly conserved residues among RAS effectors proteins are underlined in shaded sticks. The RAS protein surface is colored in gray, residues involved in intermolecular contacts with highly conserved residues among effector proteins are underlined (Asp33, Glu37, Asp38) and (Tyr64). (B) shows the computational prediction of more relevant residues for the design of a new set of peptidomimetics.

FIG. 5 shows a Western Blot of the different proteins of the KRAS signaling pathway to evaluate the synthesized therapeutic compounds IP-14-01 (P1), IP-14-02 (P2) IP-14-03 (P3), IP-14-04 (P4), IP-14-07 (P7), IP-14-08 (P8) and IP-14-09 (P9). GTPase activating protein (GAP120) was used as loading control. Test therapeutic compounds were applied to a serum starved cell culture (0.5% FCS for 24 h) of hTERT-RPE (hereinafter, also referred to as an RPE cell line) 2 h prior to EGF (50 ng/ml) treatement for 10 min. DMSO was the solvating agent to dilute the peptides and then after dilution with a cell medium, the final percentage of DMSO was 0.5%.

FIG. 6 shows a western blot conducted under the same conditions of the western blot of FIG. 5, except β-cyclodextrin was used at 0.5%, instead of DMSO to evaluate compounds IP-14-01 (P1), IP-14-02 (P2) IP-14-03 (P3), IP-14-04 (P4), IP-14-07 (P7), IP-14-08 (P8) and IP-14-09 (P9).

FIG. 7 shows Western Blots to evaluate 2018/IP-14-01 (P1) and its derived peptidomimetics, including, IPR-471 (P1.1.), IPR-472 (P1.2), IPR-473 (P1.3) and IPR-474 (P1.4). For this experiment, we followed the same protocol conditions that were used in the prior WB assays for the first generation of peptidomimetics set forth for FIG. 5. Compounds were applied to a cell culture of hTERT-RPE cells at 50 UM at 0.5% DMSO for 2 h, and then the cells were treated 10 min with EGF (50 ng/ml). We did not identify any solubility issues with these peptidometics.

FIG. 8 shows a list of therapeutic compounds for the treatment of a cancer.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human. In an embodiment, a “subject” of diagnosis or treatment is a prokaryotic or a eukaryotic cell, a tissue culture, a tissue or an animal, e.g. a mammal, including a human.

As used herein, the term “comprising” is intended to mean that the compositions and methods include the listed elements, but do not exclude other unlisted elements. “Consisting essentially of” when used to define compositions and methods, excludes other elements that alters the basic nature of the composition and/or method, but does not exclude other unlisted elements. Thus, a composition consisting essentially of the elements as defined herein would not exclude trace amounts of elements, such as contaminants from any isolation and purification methods or pharmaceutically acceptable carriers, such as phosphate buffered saline, preservatives, and the like, but would exclude additional unspecified amino acids. “Consisting of” excludes more than trace elements of other ingredients and substantial method steps for administering the compositions described herein. Embodiments defined by each of these transition terms are within the scope of this disclosure and the inventions embodied therein.

As used herein, the term “hydrate” refers to a crystal form with either a stoichiometric or non-stoichiometric amount of water incorporated into the crystal structure.

The term “alkenyl” as used herein refers to an unsaturated straight or branched hydrocarbon having at least one carbon-carbon double bond, such as a straight or branched group of 2-8 carbon atoms, referred to herein as (C2-C8) alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkoxy” as used herein refers to an alkyl group attached to an oxygen (—O-alkyl-). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-8 carbon atoms, referred to herein as (C1-C8) alkoxy. Exemplary alkoxy groups include, but are not limited to methoxy and ethoxy.

The term “alkyl” as used herein refers to a saturated straight or branched hydrocarbon, such as a straight or branched group of 1-8 carbon atoms, referred to herein as (C1-C8) alkyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl.

The term “alkynyl” as used herein refers to an unsaturated straight or branched hydrocarbon having at least one carbon-carbon triple bond, such as a straight or branched group of 2-8 carbon atoms, referred to herein as (C2-C8) alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “amide” as used herein refers to the form —NRaC(O)(Rb)— or —C(O)NRbRc, wherein Ra, Rb and Rc are each independently selected from alkyl, alkenyl, alkynyl, aryl, arylalkyl, cycloalkyl, haloalkyl, heteroaryl, heterocyclyl, and hydrogen. The amide can be attached to another group through the carbon, the nitrogen, Rb, or Rc. The amide also may be cyclic, for example Rb and Rc, may be joined to form a 3- to 8-membered ring, such as 5- or 6-membered ring. The term “amide” encompasses groups such as sulfonamide, urea, ureido, carbamate, carbamic acid, and cyclic versions thereof. The term “amide” also encompasses an amide group attached to a carboxy group, e.g., -amide-COOH or salts such as -amide-COONa, an amino group attached to a carboxy group (e.g., -amino-COOH or salts such as -amino-COONa).

The term “amine” or “amino” as used herein refers to the form —NRdRe or —N(Rd)Re—, where Rd and Re are independently selected from alkyl, alkenyl, alkynyl, aryl, arylalkyl, carbamate, cycloalkyl, haloalkyl, heteroaryl, heterocyclyl, and hydrogen. The amino can be attached to the parent molecular group through the nitrogen. The amino also may be cyclic, for example any two of Rd and Re may be joined together or with the N to form a 3- to 12-membered ring (e.g., morpholino or piperidinyl). The term amino also includes the corresponding quaternary ammonium salt of any amino group. Exemplary amino groups include alkylamino groups, wherein at least one of Rd or Re is an alkyl group. In some embodiments Rd and Re each may be optionally substituted with hydroxyl, halogen, alkoxy, ester, or amino.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to a monocyclic aromatic ring system, wherein the ring comprises 6 carbon atoms, referred to herein as “(C6) aryl.”

The term “arylalkyl” as used herein refers to an alkyl group having at least one aryl substituent (e.g., -aryl-alkyl-). Exemplary arylalkyl groups include, but are not limited to, arylalkyls having a monocyclic aromatic ring system, wherein the ring comprises 6 carbon atoms, referred to herein as “(C6) arylalkyl.”

The term “carbamate” as used herein refers to the form —Rgoc(O)N(Rh)—, —Rgoc(O)N(Rh)Ri-, or -oc(O)NRhRi, wherein Rg, Rh and Ri are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates (e.g., wherein at least one of Rg, Rh and Ri are independently selected from aryl or heteroaryl, such as pyridine, pyridazine, pyrimidine, and pyrazine).

The term “carboxy” as used herein refers to —COON or its corresponding carboxylate salts (e.g., —COONa). The term carboxy also includes “carboxycarbonyl,” e.g. a carboxy group attached to a carbonyl group, e.g., —C(O)—COOH or salts, such as —C(O)—COONa.

The term “cyano” as used herein refers to —CN.

The term “cycloalkoxy” as used herein refers to a cycloalkyl group attached to an oxygen.

The term “cycloalkyl” as used herein refers to a saturated or unsaturated cyclic, bicyclic, or bridged bicyclic hydrocarbon group of 3-12 carbons, or 3-8 carbons, referred to herein as “(C3-C8)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl saturated or unsaturated, aryl, or heterocyclyl groups.

The term “dicarboxylic acid” as used herein refers to a group containing at least two carboxylic acid groups such as saturated and unsaturated hydrocarbon dicarboxylic acids and salts thereof. Exemplary dicarboxylic acids include alkyl dicarboxylic acids. Dicarboxylic acids may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides (for example, succinic anhydride and succinimide).

The term “ester” refers to the structure —C(O)O—, —C(O)O-Rj-, —RkC(O)O-Rj-, or —RkC(O)O—, where O is not bound to hydrogen, and Rj and Rk can independently be selected from alkoxy, aryloxy, alkyl, alkenyl, alkynyl, amide, amino, aryl, arylalkyl, cycloalkyl, ether, haloalkyl, heteroaryl, and heterocyclyl. Rk can be a hydrogen, but Rj cannot be hydrogen. The ester may be cyclic, for example the carbon atom and Rj, the oxygen atom and Rk, or Rj and Rk may be joined to form a 3- to 12-membered ring. Exemplary esters include, but are not limited to, alkyl esters wherein at least one of Rj or Rk is alkyl, such as —O—C(O)-alkyl, —C(O)—O-alkyl-, and -alkyl-C(O)—O— alkyl-. Exemplary esters also include aryl or heteoraryl esters, e.g. wherein at least one of Rj or Rk is a heteroaryl group such as pyridine, pyridazine, pyrimidine and pyrazine, such as a nicotinate ester. Exemplary esters also include reverse esters having the structure -RkC(O)O—, where the oxygen is bound to the parent molecule. Exemplary reverse esters include succinate, D-argininate, L-argininate, L-lysinate and D-lysinate. Esters also include carboxylic acid anhydrides and acid halides.

The terms “halo” or “halogen” as used herein refer to F, Cl, Br, or I.

The term “haloalkyl” as used herein refers to an alkyl group substituted with one or more halogen atoms. “Haloalkyls” also encompass alkenyl or alkynyl groups substituted with one or more halogen atoms.

The term “heteroaryl” as used herein refers to a mono-, bi-, or multi-cyclic, aromatic ring system containing one or more heteroatoms, for example 1-3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C2-C5)heteroaryl.”

The terms “heterocycle,” “heterocyclyl,” or “heterocyclic” as used herein refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure —C(O)—Rn (such as acetyl, —C(O)CH3) or —Rn—C(O)-Ro-. The ketone can be attached to another group through Rn or Ro. Rn or Ro can be alkyl, alkenyl, alkynyl, cycloalkyl, heterocyclyl or aryl, or Rn or Ro can be joined to form a 3- to 12-membered ring.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “N-protecting group” refers to groups intended to protect an amino group against undesirable reactions during synthetic procedures. Commonly used N-protecting groups are disclosed in Greene, “Protective Groups in Organic Synthesis,” 4^{th} Edition (John Wiley & Sons, Hoboken, N J, 2006), which is incorporated herein by reference. N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, alkaryl groups such as benzyl, triphenylmethyl, benzyloxymethyl, and the like and silyl groups such as trimethylsilyl, and the like. Preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “phenyl” as used herein refers to a 6-membered carbocyclic aromatic ring. The phenyl group can also be fused to a cyclohexane or cyclopentane ring. Phenyl can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide and thioketone.

The term “thioalkyl” as used herein refers to an alkyl group attached to a sulfur (—S-alkyl-).

The term “acetylation” or in IUPAC nomenclature “ethanoylation” refers to a reaction that introduces an acetyl functional group into a chemical compound. In contrast, deacetylation refers to the removal of an acetyl group.

“Alkyl,” “alkenyl,” “alkynyl”, “alkoxy”, “amino” and “amide” groups can be optionally substituted with or interrupted by or branched with at least one group selected from alkoxy, aryloxy, alkyl, alkenyl, alkynyl, amide, amino, aryl, arylalkyl, carbamate, carbonyl, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide, thioketone, ureido and N. The substituents may be branched to form a substituted or unsubstituted heterocycle or cycloalkyl.

As used herein, a suitable substitution on an optionally substituted substituent refers to a group that does not nullify the synthetic or pharmaceutical utility of the compounds of the present disclosure or the intermediates useful for preparing them. Examples of suitable substitutions include, but are not limited to: C1-8 alkyl, alkenyl or alkynyl; C1-6 aryl, C7-5 heteroaryl; C3-7 cycloalkyl; C1-8 alkoxy; C6 aryloxy; —CN; —OH; oxo; halo, carboxy; amino, such as —NH(C1-8 alkyl), —N(C1-8alkyl)2, —NH((C6)aryl), or —N((C6)aryl)2; formyl; ketones, such as —CO(C1-8 alkyl), —CO((C6aryl) esters, such as —CO2(C1-8 alkyl) and —CO2 (C6aryl). One of skill in art can readily choose a suitable substitution based on the stability and pharmacological and synthetic activity of the compound of the present disclosure.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

In an embodiment, a “pharmaceutical composition” is intended to include the combination of an active agent, such as a therapeutic compound of the present invention, with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present disclosure that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the present disclosure. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of the present disclosure. The present disclosure encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangements of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein may exist as tautomers and both tautomeric forms are intended to be encompassed by the scope of the present disclosure, even though only one tautomeric structure is depicted.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

In an embodiment, “an effective amount” refers, without limitation, to the amount of the defined compound sufficient to achieve the desired therapeutic result. In an embodiment, that result can be effective cancer treatment.

In an embodiment, as used herein, the terms “treating,” “treatment” and the like are used herein, without limitation, to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

As used herein, the term “recombinant” refers to polypeptides or polynucleotides that do not exist naturally and which may be created by combining polynucleotides or polypeptides in arrangements that would not normally occur together. The term can refer to a polypeptide produced through a biological host, selected from a mammalian expression system, an insect cell expression system, a yeast expression system, and a bacterial expression system.

As used herein, the term “antibody” refers to a polypeptide or a polypeptide complex that specifically recognizes and binds to an antigen through one or more immunoglobulin variable regions. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding and is encoded by the variable domain. An antibody can be a whole antibody, an antigen binding fragment or a single chain thereof.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to variable domains of the light and heavy chain respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain VL-CL joined to VH-CH1 by a disulfide bond. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

Accordingly, in either aspect of the invention, the term antibody also embraces minibodies, scFvs, diabodies, triabodies and the like. ScFvs and Diabodies are small bivalent biospecific antibody fragments with high avidity and specificity. Their high signal to noise ratio is typically better due to a better specificity and fast blood clearance increasing their potential for diagnostic and therapeutic targeting of specific antigen (Sundaresan et al., J Nucl Med 44:1962-9 (2003). In addition, these antibodies are advantageous because they can be engineered if necessary as different types of antibody fragments ranging from a small single chain Fv (scFv) to an intact IgG with varying isoforms (Wu & Senter, Nat. Biotechnol. 23:1137-1146 (2005)). In some embodiments, the antibody fragment is part of a scFv-scFv or diabody. In some embodiments, in either aspect, the invention provides high avidity antibodies for use according to the invention.

The terms “antibody fragment” or “antigen-binding fragment” are used with reference to a portion of an antibody, such as Fab′, Fab, Fv, scFv and the like. Regardless of structure, an antibody fragment binds with the same antigen that is recognized by the intact antibody. The term “antibody fragment” also includes diabodies and any synthetic or genetically engineered proteins comprising immunoglobulin variable regions that act like an antibody by binding to a specific antigen to form a complex.

The term “antigen-binding fragment” or “Fab” refers to a region on an antibody that binds to antigens. It includes one constant and one variable domain of each of the heavy and the light chain (i.e. four domains: VH, CH1, VL and CL1.). The variable domain contains the paratope (the antigen-binding site), that includes a set of complementary determining regions at the amino terminal end of the monomer. Each arm of the Y thus binds an epitope on the antigen.

The term “Fc region” or “fragment crystallizable region” refers to the tail region of an antibody CH2-CH3 that interacts with cell surface receptors called Fc receptors and some proteins of the complement system. This “effector function” allows antibodies to activate the immune system leading to cellular cytotoxicity (ADCC), antibody-dependent cellular phagocytosis (ADCP), and/or complement dependent cytotoxicity (CDC). ADCC and ADCP are mediated through the binding of the Fc to Fc receptors on the surface of cells of the immune system. CDC is mediated through the binding of the Fc with proteins of the complement system, (e.g. C1q).

In IgG, IgA and IgD antibody isotypes, the Fc region has two identical protein fragments, derived from the second and third constant domains of the antibody's two heavy chains. IgM and IgE Fc regions have three heavy chain constant domains (CH domains 2-4) in each polypeptide chain whereas IgG is composed of 2 CH domains, 2 and 3. The Fc regions of IgGs bear a highly conserved N-glycosylation site. Glycosylation of the Fc fragment is essential for Fc receptor-mediated activity. The N-glycans attached to this site are predominantly core-fucosylated diantennary structures of the complex type. In addition, small amounts of these N-glycans also bear bisecting GlcNAc and α-2,6 linked sialic acid residues.

The term “scFv” or “scFv fragment antibody” refers to a small molecular antibody, consisting of VH and VL domains, either in the configuration of VL-VH or VH-VL, with a linker region between them. The scFv fragment antibody can more easily penetrate blood vessel wall and the solid tumor, which makes it a preferred carrier of targeting drugs.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

The present therapeutic peptide can have amino acid additions, deletions, or substitutions. A modified amino acid sequence is a sequence that is different from the native amino acid sequence due to a deletion, an insertion, a non-conservative or conservative substitution or combinations thereof of one or more amino acid residues. In one embodiment, the modification is a point mutation. In one aspect, the modified therapeutic peptide does not have a naturally occurring sequence.

The amino acid substitutions may be conservative or non-conservative. A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The most commonly occurring exchanges are Ala/Ser, Val/Ile, Asp/Glu, Thr/Ser, Ala/Gly, Ala/Thr, Ser/Asn, Ala/Val, Ser/Gly, Ala/Pro, Lys/Arg, Asp/Asn, Leu/Ile, Leu/Val, Ala/Glu and Asp/Gly, in both directions. Amino acid exchanges in proteins and peptides, which do not generally alter the activity of the proteins or peptides, are known in the art (H. Neurath, R. L. Hill, The Proteins, Academic Press, New York, 1979).

The term “derivative of a peptide” refers to a peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine.

Alternatively, the amino acid can be a modified amino acid residue and/or can be an amino acid that is modified by post-translation modification (e.g., acetylation, amidation, formylation, hydroxylation, methylation, phosphorylation or sulfatation). A non-naturally occurring amino acid can be an “unnatural” amino acid, which can be used in a therapeutic compound of the present invention.

The term non-natural or unusual amino acids includes those can be built into synthetic peptides. These include D-amino acids, homo amino acids, beta-homo amino acids, N-methyl amino acids, alpha-methyl amino acids, non-natural side chain variant amino acids and other unusual amino acids. D-amino acids involve the mirror image of the naturally occurring L-isomers. Homo-amino acids is an amino acid that includes the addition of a methylene (CH²) group to the α-carbon of an amino acid. Beta-homo-amino acids are analogs of standard amino acids in which the carbon skeleton has been lengthened by insertion of one carbon atom immediately after the acid group. N-methyl amino acids are amino acids that carry a methyl group at the nitrogen instead of a proton. Alpha-methyl amino acids are natural amino acid variants, in which the proton on the α-carbon atom of the natural original (in between the amino and carboxy group) has been substituted by a methyl group. Unusual amino acids occur most frequently in microbial peptides and proteins and are formed posttranslationally. The unusually amino acids often contribute to the special bioactivity of these peptides. Additionally, an amino acid can be synthetic non-natural.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Nucleic acid” refers to deoxyribonucleotides or ribonucleotides and polymers thereof in either single- or double-stranded form and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

As used herein, the term “prevention” means all of the actions by which the occurrence of the disease is restrained or retarded.

As used herein, the term “treatment” means all of the actions by which the symptoms of the disease have been alleviated, improved or ameliorated. In the present specification, “treatment” means that the symptoms of cancer, neurodegeneration, or infectious disease are alleviated, improved or ameliorated by administration of the antibodies disclosed herein.

The term “administration” refers to the introduction of an amount of a predetermined substance into a patient by a certain suitable method. The composition disclosed herein may be administered via any of the common routes, as long as it is able to reach a desired tissue, for example, but is not limited to, intraperitoneal, intravenous, intramuscular, subcutaneous, intradermal, oral, topical, intranasal, intrapulmonary, or intrarectal administration. However, since peptides are digested upon oral administration, active ingredients of a composition for oral administration should be coated or formulated for protection against degradation in the stomach.

The term “subject” refers to those suspected of having or diagnosed with cancer, a neurodegenerative or an infectious disease. However, any subject to be treated with the pharmaceutical composition disclosed herein is included without limitation. The pharmaceutical composition including an anti-DLL3 antibody disclosed herein is administered to a subject suspected of having cancer, a neurodegenerative or an infectious disease.

The term “cancer” refers to human cancers and carcinomas, sarcomas, adenocarcinomas, etc., including solid tumors, kidney, breast, lung, kidney, bladder, urinary tract, urethra, penis, vulva, vagina, cervical, colon, ovarian, prostate, pancreas, stomach, brain, head and neck, skin, uterine, testicular, esophagus, and liver cancer. Additional cancers include, for example, Hodgkin's Disease, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, thyroid cancer, neuroblastoma, esophageal cancer, genitourinary tract cancer, malignant hypercalcemia, cervical cancer, endometrial cancer, and adrenal cortical cancer.

In any of the embodiments above, one or more cancer therapies, e.g., chemotherapy, radiation therapy, immunotherapy, surgery, or hormone therapy can be co-administered further with an antibody of the invention.

In one embodiment, the therapeutic compound is an alkylating agent: nitrogen mustards, nitrosoureas, tetrazines, aziridines, cisplatins and derivatives, and non-classical alkylating agents. Nitrogen mustards include mechlorethamine, cyclophosphamide, melphalan, chlorambucil, ifosfamide and busulfan. Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide. Aziridines include thiotepa, mytomycin and diaziquone (AZQ). Cisplatin and derivatives include cisplatin, carboplatin and oxaliplatin. In one embodiment the chemotherapeutic reagent is an anti-metabolites: the anti-folates (e.g., methotrexate), fluoropyrimidines (e.g., fluorouracil and capecitabine), deoxynucleoside analogues and thiopurines. In another embodiment the chemoptheraputic reagent is an anti-microtubule agent such as vinca alkaloids (e.g., vincristine and vinblastine) and taxanes (e.g., paclitaxel and docetaxel). In another embodiment the chemotherapeutic reagent is a topoisomerase inhibitor or a cytotoxic antibiotic such as doxorubicin, mitoxantrone, bleomycin, actinomycin, and mitomycin.

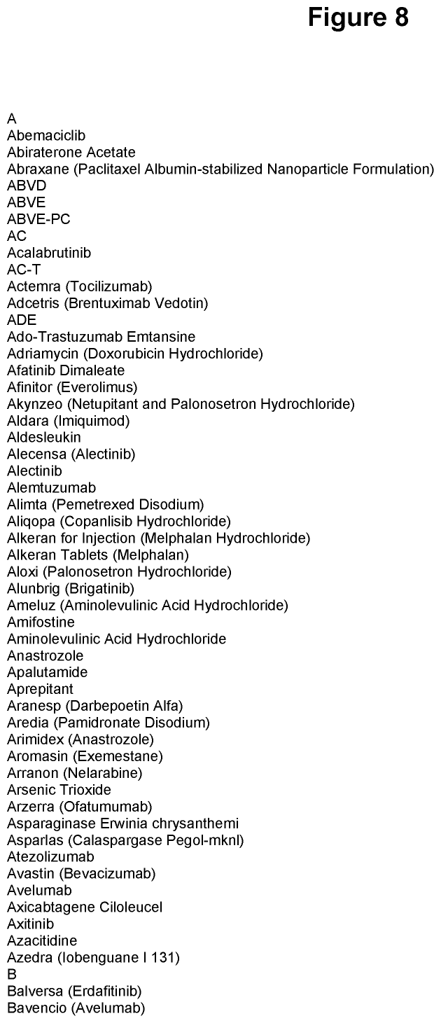

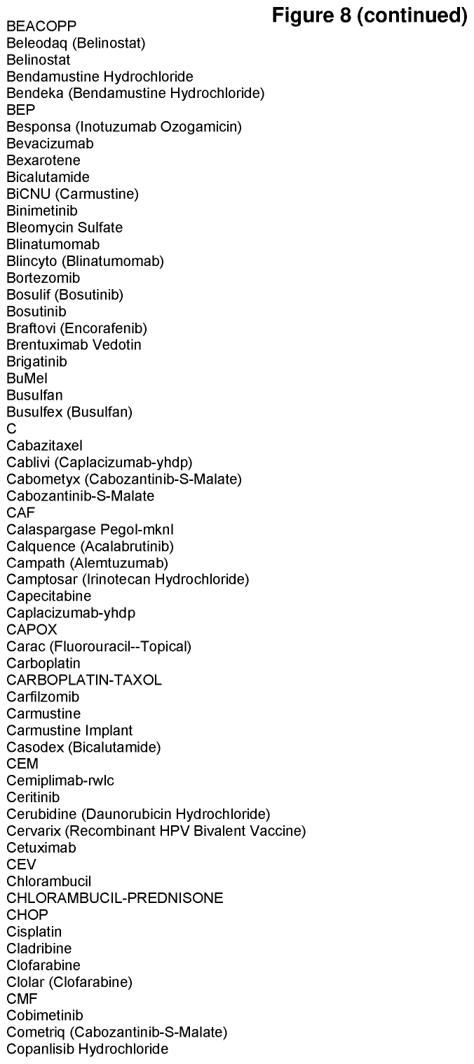

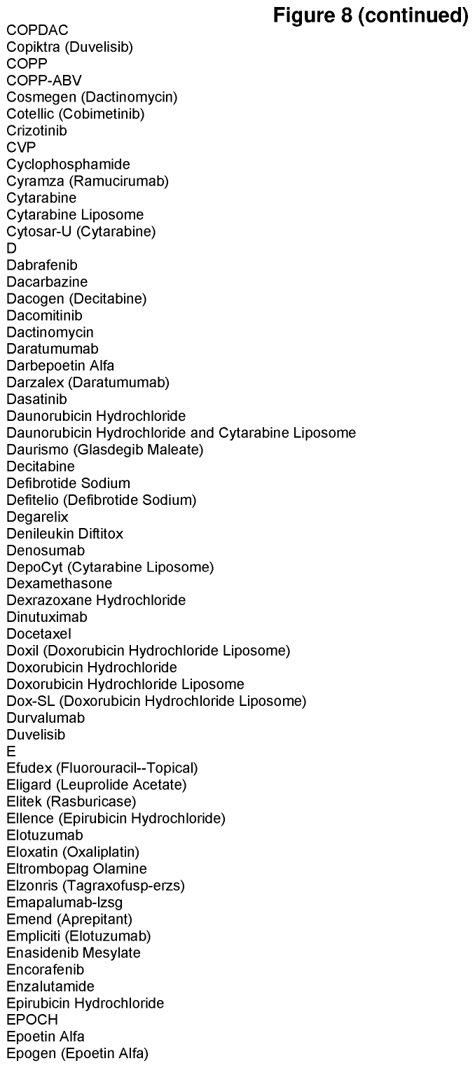

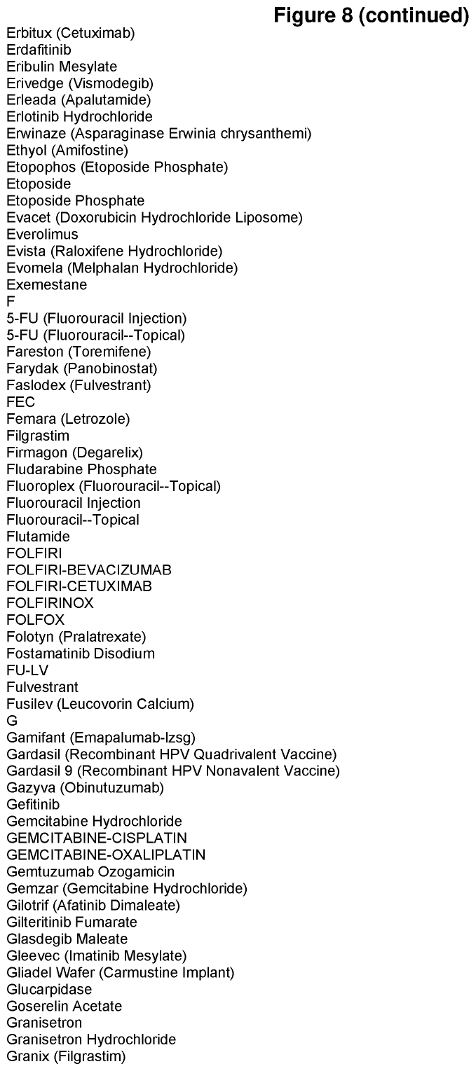

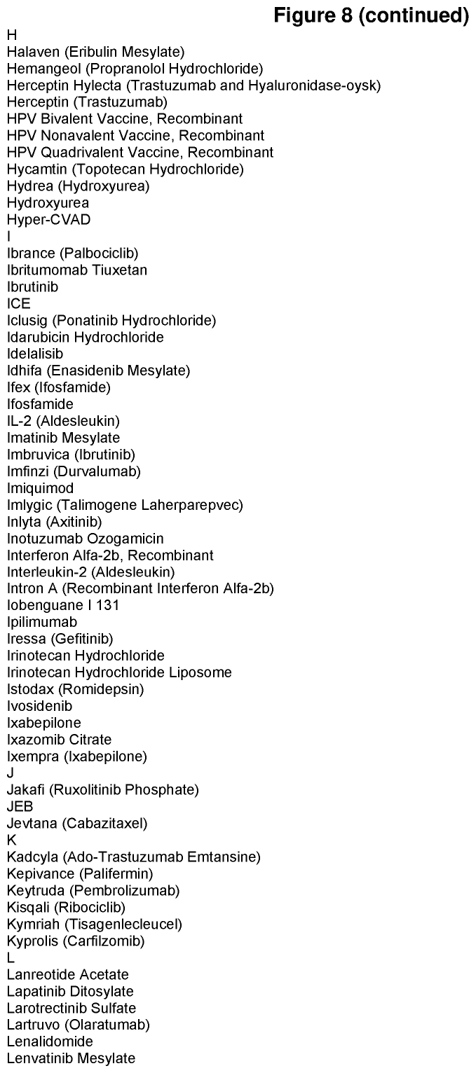

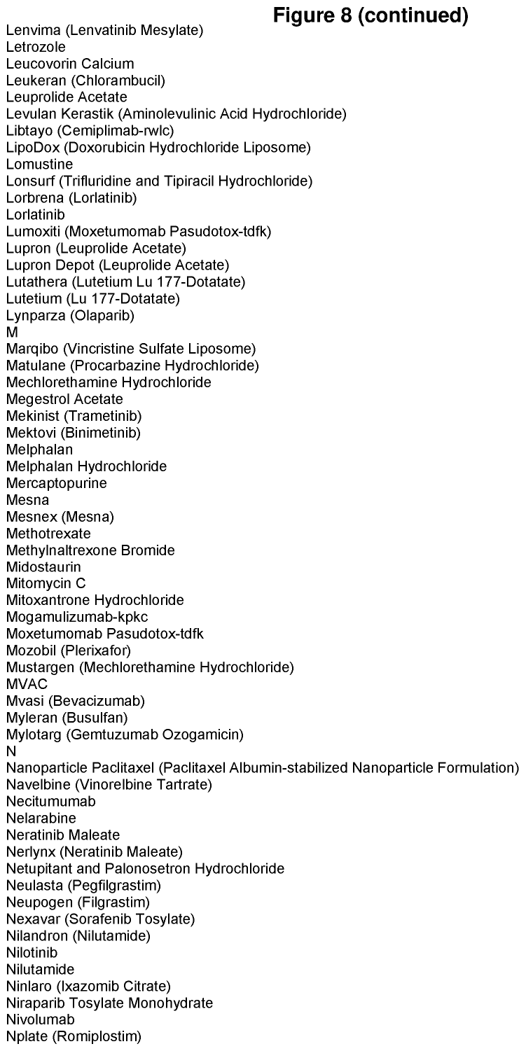

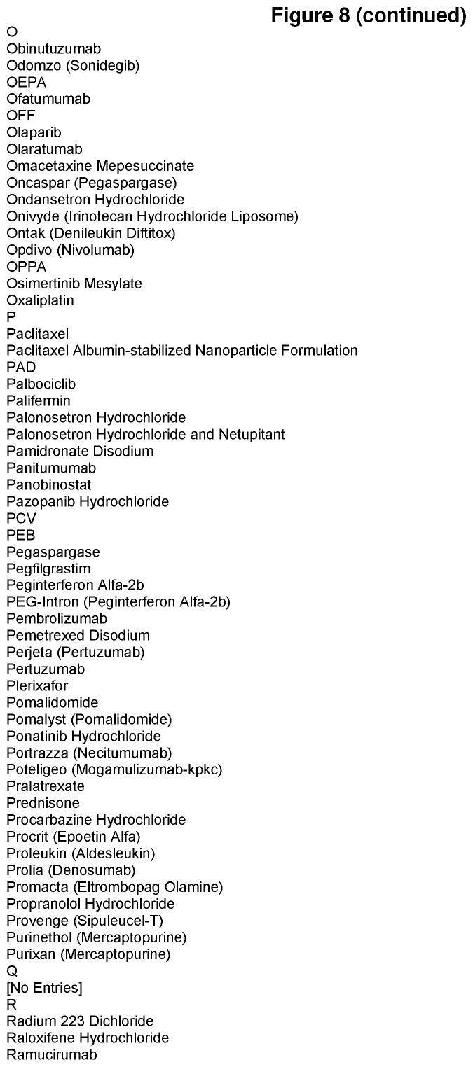

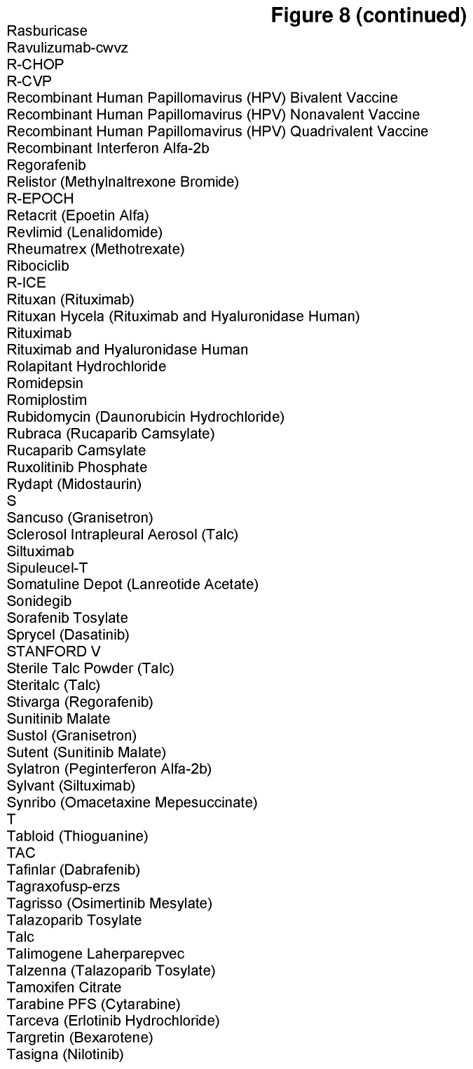

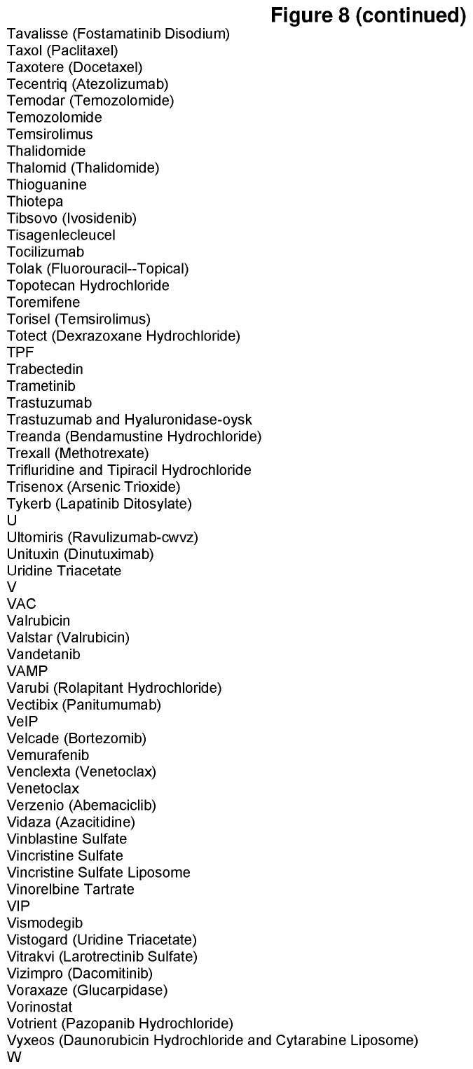

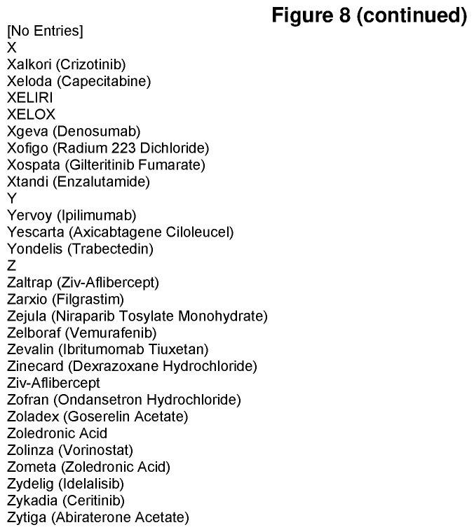

In another embodiment, a therapeutic compound is one identified in FIG. 8.

The contacting of the patient with a therapeutic compound, can be by administering the antibody to the patient intravenously, intraperitoneally, intramuscularly, intratumorally, or intradermally. In some embodiments the therapeutic compound is co-administered with a cancer therapy agent.

The term “formulation” as used herein refers to the therapeutic compounds disclosed herein and excipients combined together which can be administered and has the ability to bind to the corresponding receptors and initiate a signal transduction pathway resulting in the desired activity. The formulation can optionally comprise other agents.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

As the patients and subjects of the invention method are, in addition to humans, veterinary subjects, formulations suitable for these subjects are also appropriate. Such subjects include livestock and pets as well as sports animals such as horses, greyhounds, and the like.

For use as treatment of human and animal subjects, the therapeutic compound of the invention can be formulated as pharmaceutical or veterinary compositions. Depending on the subject to be treated, the mode of administration, and the type of treatment desired (e.g., prevention, prophylaxis, or therapy) the therapeutic compound are formulated in ways consonant with these parameters. A summary of such techniques is found in Remington: The Science and Practice of Pharmacy, 21” Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

The therapeutic compound described herein may be present in amounts totaling 1-95% by weight of the total weight of the pharmaceutical composition. The pharmaceutical composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurcthral, intrathecal, epidural, aural, or ocular administration, or by injection, inhalation, or direct contact with the nasal, genitourinary, gastrointestinal, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

### DETAILED DESCRIPTION

Embodiments of the invention include methods of treating, monitoring and preventing cancer, including refractory cancer, using a therapeutic compound, pharmaceutical compositions thereof, and articles of manufacture.

### Methods of Treatment

Another aspect of the present application relates to a method for treating a cell proliferative disorder. The method comprises administering to a subject in need thereof an effective amount of a therapeutic compound according to the present disclosure. In another aspect, a method for treating a cell proliferative disorder comprises administering to a subject in need thereof an effective amount of a therapeutic compound according to the present disclosure.

Any suitable route or mode of administration can be employed for providing the patient with a therapeutically or prophylactically effective dose of a therapeutic compound. Exemplary routes or modes of administration include parenteral {e.g., intravenous, intraarterial, intramuscular, subcutaneous, intratumoral), oral, topical (nasal, transdermal, intradermal or intraocular), mucosal {e.g., nasal, sublingual, buccal, rectal, vaginal), inhalation, intralymphatic, intraspinal, intracranial, intraperitoneal, intratracheal, intravesical, intrathecal, enteral, intrapulmonary, intralymphatic, intracavital, intraorbital, intracapsular and transurethral, as well as local delivery by catheter or stent.

A pharmaceutical composition comprising a therapeutic compound in accordance with the present disclosure can be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions can include suitable solid or gel phase carriers or excipients. Exemplary carriers or excipients include calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases it will be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the therapeutic agents.

In an embodiment, a therapeutic compound can be incorporated into a pharmaceutical composition suitable for parenteral administration. Suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05%>polysorbate-80 (optimally 0.005-0.0 1%). Additional surfactants include but are not limited to polysorbate 20 and BRIJ surfactants.

A pharmaceutical composition comprising a therapeutic compound can be lyophilized and stored as sterile powders, preferably under vacuum, and then reconstituted in bacteriostatic water (containing, for example, benzyl alcohol preservative) or in sterile water prior to injection. Pharmaceutical compositions can be formulated for parenteral administration by injection e.g., by bolus injection or continuous infusion.

The therapeutic compounds in the pharmaceutical compositions may be formulated in a “therapeutically effective amount” or a “prophylactically effective amount”. A “therapeutically effective amount” refers to an amount effective, at dosages and for periods of time necessary, to achieve the desired therapeutic result. A therapeutically effective amount of the recombinant vector may vary depending on the condition to be treated, the severity and course of the condition, the mode of administration, whether the antibody or agent is administered for preventive or therapeutic purposes, the bioavailability of the particular agent(s), the ability of the trispecific antibody to elicit a desired response in the individual, previous therapy, the age, weight and sex of the patient, the patient's clinical history and response to the antibody, the type of the trispecific antibody used, discretion of the attending physician, etc. A therapeutically effective amount is also one in which any toxic or detrimental effects of the recombinant vector is outweighed by the therapeutically beneficial effects. A “prophylactically effective amount” refers to an amount effective, at dosages and for periods of time necessary, to achieve the desired prophylactic result.

A therapeutic compound is suitably administered to the patient at one time or over a series of treatments and may be administered to the patient at any time from diagnosis onwards. A therapeutic compound may be administered as the sole treatment or in conjunction with other drugs or therapies useful in treating the condition in question.

In an embodiment, a therapeutic compound of the present invention comprises peptidomimetic P1.3, also referred to as IPR-473 below, or a derivative of P1.3, which has the following structure:

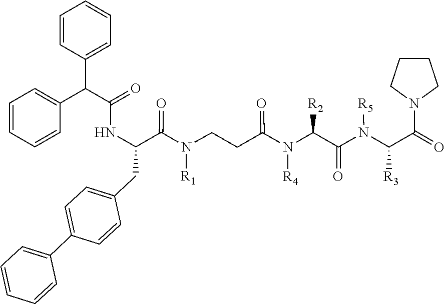

wherein, R1 comprises one of the following:
   CH3, CH2-CH3, CH2-CH2-CH3, CH2-CH2-CH2-CH3 or CH2-CH2-CH2-Hexyl;
and R2 comprises one of the following:
   Sidechains corresponding to Dab, Cyclohexylglicine, Asn, Cys, Gly, Ile, Thr or Lys
and R3 comprises one of the following:
   Sidechains corresponding to Leu, Ala, Gly, Phe, Ile, Tyr or Val
and R4 comprises one of the following:
   H, CH3, or alkyl group
and R5 comprises one of the following:
   H, CH3, or alkyl group

In an embodiment, a therapeutic compound of the present invention comprises one of the following peptidomimetics, wherein P1.3 is the structure set forth above wherein the specific R1, R2 and R3 of each peptidomimetic is set forth below in Table 1 along with the associated KRAS-GTPase Docking Score of the specific peptidomimetic are set forth in Table 1 below (comprising 140 variants of P1.3):

TABLE 1

| ID | R1 | R2 | R3 | Docking Score |
|---|---|---|---|---|
| P1 | — | — | — | −4.958 |
| P2 | — | — | — | −5.602 |
| P3 | — | — | — | −5.591 |
| P4 | — | — | — | −5.23 |
| P5 | — | — | — | −5.27 |
| P6 | — | — | — | −4.172 |
| P7 | — | — | — | −5.74 |
| P8 | — | — | — | −4.958 |
| P9 | — | — | — | −4.958 |
| P1.1 | — | — | — | −4.301 |
| P1.2 | — | — | — | −3.722 |
| P1.3 | — | — | — | −3.887 |
| P1.4 | — | — | — | −5.454 |
| P1.3.1 | CH2—CH3 | Ciclohexylglicine | Gly | −3.78 |
| P1.3.2 | CH2—CH3 | Ciclohexylglicine | Phe | 1.386 |
| P1.3.3 | CH2—CH3 | Ciclohexylglicine | Ile | −3.104 |
| P1.3.4 | CH2—CH3 | Ciclohexylglicine | Tyr | −1.802 |
| P1.3.5 | CH2—CH3 | Ciclohexylglicine | Val | −1.895 |
| P1.3.6 | CH2—CH3 | Asn | Gly | −2.973 |
| P1.3.7 | CH2—CH3 | Asn | Phe | −3.853 |
| P1.3.8 | CH2—CH3 | Asn | Ile | −1.114 |
| P1.3.9 | CH2—CH3 | Asn | Tyr | −3.718 |
| P1.3.10 | CH2—CH3 | Asn | Val | −4.482 |
| P1.3.11 | CH2—CH3 | Cys | Gly | −2.612 |
| P1.3.12 | CH2—CH3 | Cys | Phe | −1.893 |
| P1.3.13 | CH2—CH3 | Cys | Ile | −2.659 |
| P1.3.14 | CH2—CH3 | Cys | Tyr | −3.272 |
| P1.3.15 | CH2—CH3 | Cys | Val | −3.839 |
| P1.3.16 | CH2—CH3 | Gly | Gly | −3.058 |
| P1.3.17 | CH2—CH3 | Gly | Phe | — |
| P1.3.18 | CH2—CH3 | Gly | Ile | 2.297 |
| P1.3.19 | CH2—CH3 | Gly | Tyr | −0.731 |
| P1.3.20 | CH2—CH3 | Gly | Val | — |
| P1.3.21 | CH2—CH3 | Ile | Gly | −2.898 |
| P1.3.22 | CH2—CH3 | Ile | Phe | −2.387 |
| P1.3.23 | CH2—CH3 | Ile | Ile | −4.156 |
| P1.3.24 | CH2—CH3 | Ile | Tyr | −2.989 |
| P1.3.25 | CH2—CH3 | Ile | Val | −2.802 |
| P1.3.26 | CH2—CH3 | Thr | Gly | −3.489 |
| P1.3.27 | CH2—CH3 | Thr | Phe | −1.467 |
| P1.3.28 | CH2—CH3 | Thr | Ile | −2.16 |
| P1.3.29 | CH2—CH3 | Thr | Tyr | −3.713 |
| P1.3.30 | CH2—CH3 | Thr | Val | −2.914 |
| P1.3.31 | CH2—CH3 | Lys | Gly | −5.981 |
| P1.3.32 | CH2—CH3 | Lys | Phe | −2.746 |
| P1.3.33 | CH2—CH3 | Lys | Ile | −2.776 |
| P1.3.34 | CH2—CH3 | Lys | Tyr | −4.495 |
| P1.3.35 | CH2—CH3 | Lys | Val | −3.326 |
| P1.3.36 | CH2—CH2—CH3 | Ciclohexylglicine | Gly | −0.609 |
| P1.3.37 | CH2—CH2—CH3 | Ciclohexylglicine | Phe | — |
| P1.3.38 | CH2—CH2—CH3 | Ciclohexylglicine | Ile | −2.035 |
| P1.3.39 | CH2—CH2—CH3 | Ciclohexylglicine | Tyr | −2.775 |
| P1.3.40 | CH2—CH2—CH3 | Ciclohexylglicine | Val | 1.703 |
| P1.3.41 | CH2—CH2—CH3 | Asn | Gly | −3.361 |
| P1.3.42 | CH2—CH2—CH3 | Asn | Phe | −1.995 |
| P1.3.43 | CH2—CH2—CH3 | Asn | Ile | 2.834 |
| P1.3.44 | CH2—CH2—CH3 | Asn | Tyr | −2.765 |
| P1.3.45 | CH2—CH2—CH3 | Asn | Val | −1.928 |
| P1.3.46 | CH2—CH2—CH3 | Cys | Gly | −2.671 |
| P1.3.47 | CH2—CH2—CH3 | Cys | Phe | −2.687 |
| P1.3.48 | CH2—CH2—CH3 | Cys | Ile | −2.767 |
| P1.3.49 | CH2—CH2—CH3 | Cys | Tyr | −3.263 |
| P1.3.50 | CH2—CH2—CH3 | Cys | Val | 0.783 |
| P1.3.51 | CH2—CH2—CH3 | Gly | Gly | −2.614 |
| P1.3.52 | CH2—CH2—CH3 | Gly | Phe | −0.33 |
| P1.3.53 | CH2—CH2—CH3 | Gly | Ile | −3.065 |
| P1.3.54 | CH2—CH2—CH3 | Gly | Tyr | −3.753 |
| P1.3.55 | CH2—CH2—CH3 | Gly | Val | −2.968 |
| P1.3.56 | CH2—CH2—CH3 | Ile | Gly | −3.512 |
| P1.3.57 | CH2—CH2—CH3 | Ile | Phe | 2.108 |
| P1.3.58 | CH2—CH2—CH3 | Ile | Ile | −2.63 |
| P1.3.59 | CH2—CH2—CH3 | Ile | Tyr | −3.554 |
| P1.3.60 | CH2—CH2—CH3 | Ile | Val | −3.896 |
| P1.3.61 | CH2—CH2—CH3 | Thr | Gly | −2.326 |
| P1.3.62 | CH2—CH2—CH3 | Thr | Phe | −3.148 |
| P1.3.63 | CH2—CH2—CH3 | Thr | Ile | −2.746 |
| P1.3.64 | CH2—CH2—CH3 | Thr | Tyr | −3.142 |
| P1.3.65 | CH2—CH2—CH3 | Thr | Val | −2.534 |
| P1.3.66 | CH2—CH2—CH3 | Lys | Gly | −0.568 |
| P1.3.67 | CH2—CH2—CH3 | Lys | Phe | −1.237 |
| P1.3.68 | CH2—CH2—CH3 | Lys | Ile | −2.981 |
| P1.3.69 | CH2—CH2—CH3 | Lys | Tyr | −5.598 |
| P1.3.70 | CH2—CH2—CH3 | Lys | Val | −6.366 |
| P1.3.71 | CH2—CH2—CH2—CH3 | Ciclohexylglicine | Gly | −2.216 |
| P1.3.72 | CH2—CH2—CH2—CH3 | Ciclohexylglicine | Phe | −1.641 |
| P1.3.73 | CH2—CH2—CH2—CH3 | Ciclohexylglicine | Ile | −1.563 |
| P1.3.74 | CH2—CH2—CH2—CH3 | Ciclohexylglicine | Tyr | −1.68 |
| P1.3.75 | CH2—CH2—CH2—CH3 | Ciclohexylglicine | Val | −0.456 |
| P1.3.76 | CH2—CH2—CH2—CH3 | Asn | Gly | −3.468 |
| P1.3.77 | CH2—CH2—CH2—CH3 | Asn | Phe | −1.989 |
| P1.3.78 | CH2—CH2—CH2—CH3 | Asn | Ile | 0.781 |
| P1.3.79 | CH2—CH2—CH2—CH3 | Asn | Tyr | −3.643 |
| P1.3.80 | CH2—CH2—CH2—CH3 | Asn | Val | −2.758 |
| P1.3.81 | CH2—CH2—CH2—CH3 | Cys | Gly | −3.091 |
| P1.3.82 | CH2—CH2—CH2—CH3 | Cys | Phe | −2.535 |
| P1.3.83 | CH2—CH2—CH2—CH3 | Cys | Ile | −2.784 |
| P1.3.84 | CH2—CH2—CH2—CH3 | Cys | Tyr | −3.584 |
| P1.3.85 | CH2—CH2—CH2—CH3 | Cys | Val | −3.505 |
| P1.3.86 | CH2—CH2—CH2—CH3 | Gly | Gly | −3.517 |
| P1.3.87 | CH2—CH2—CH2—CH3 | Gly | Phe | — |
| P1.3.88 | CH2—CH2—CH2—CH3 | Gly | Ile | 4.701 |
| P1.3.89 | CH2—CH2—CH2—CH3 | Gly | Tyr | −2.567 |
| P1.3.90 | CH2—CH2—CH2—CH3 | Gly | Val | 0.424 |
| P1.3.91 | CH2—CH2—CH2—CH3 | Ile | Gly | −2.976 |
| P1.3.92 | CH2—CH2—CH2—CH3 | Ile | Phe | −3.04 |
| P1.3.93 | CH2—CH2—CH2—CH3 | Ile | Ile | −2.522 |
| P1.3.94 | CH2—CH2—CH2—CH3 | Ile | Tyr | −3.637 |
| P1.3.95 | CH2—CH2—CH2—CH3 | Ile | Val | −2.379 |
| P1.3.96 | CH2—CH2—CH2—CH3 | Thr | Gly | −3.038 |
| P1.3.97 | CH2—CH2—CH2—CH3 | Thr | Phe | −2.347 |
| P1.3.98 | CH2—CH2—CH2—CH3 | Thr | Ile | −2.691 |
| P1.3.99 | CH2—CH2—CH2—CH3 | Thr | Tyr | −4.508 |
| P1.3.100 | CH2—CH2—CH2—CH3 | Thr | Val | 1.794 |
| P1.3.101 | CH2—CH2—CH2—CH3 | Lys | Gly | −6.152 |
| P1.3.102 | CH2—CH2—CH2—CH3 | Lys | Phe | −1.084 |
| P1.3.103 | CH2—CH2—CH2—CH3 | Lys | Ile | −3.723 |
| P1.3.104 | CH2—CH2—CH2—CH3 | Lys | Tyr | −4.408 |
| P1.3.105 | CH2—CH2—CH2—CH3 | Lys | Val | −1.654 |
| P1.3.106 | CH2—CH2—CH2— | Ciclohexylglicine | Gly | −1.282 |
| | Hexyl | | | |
| P1.3.107 | CH2—CH2—CH2— | Ciclohexylglicine | Phe | — |
| | Hexyl | | | |
| P1.3.108 | CH2—CH2—CH2— | Ciclohexylglicine | Ile | −0.863 |
| | Hexyl | | | |
| P1.3.109 | CH2—CH2—CH2— | Ciclohexylglicine | Tyr | — |
| | Hexyl | | | |
| P1.3.110 | CH2—CH2—CH2— | Ciclohexylglicine | Val | 0.053 |
| | Hexyl | | | |
| P1.3.111 | CH2—CH2—CH2— | Asn | Gly | −1.683 |
| | Hexyl | | | |
| P1.3.112 | CH2—CH2—CH2— | Asn | Phe | −2.231 |
| | Hexyl | | | |
| P1.3.113 | CH2—CH2—CH2— | Asn | Ile | −1.732 |
| | Hexyl | | | |
| P1.3.114 | CH2—CH2—CH2— | Asn | Tyr | — |
| | Hexyl | | | |
| P1.3.115 | CH2—CH2—CH2— | Asn | Val | −0.644 |
| | Hexyl | | | |
| P1.3.116 | CH2—CH2—CH2— | Cys | Gly | −1.871 |
| | Hexyl | | | |
| P1.3.117 | CH2—CH2—CH2— | Cys | Phe | −1.654 |
| | Hexyl | | | |
| P1.3.118 | CH2—CH2—CH2— | Cys | Ile | 1.32 |
| | Hexyl | | | |
| P1.3.119 | CH2—CH2—CH2— | Cys | Tyr | −3.525 |
| | Hexyl | | | |
| P1.3.120 | CH2—CH2—CH2— | Cys | Val | −1.609 |
| | Hexyl | | | |
| P1.3.121 | CH2—CH2—CH2— | Gly | Gly | −0.991 |
| | Hexyl | | | |
| P1.3.122 | CH2—CH2—CH2— | Gly | Phe | — |
| | Hexyl | | | |
| P1.3.123 | CH2—CH2—CH2— | Gly | Ile | −0.5 |
| | Hexyl | | | |
| P1.3.124 | CH2—CH2—CH2— | Gly | Tyr | −2.485 |
| | Hexyl | | | |
| P1.3.125 | CH2—CH2—CH2— | Gly | Val | — |
| | Hexyl | | | |
| P1.3.126 | CH2—CH2—CH2— | Ile | Gly | 1.911 |
| | Hexyl | | | |
| P1.3.127 | CH2—CH2—CH2— | Ile | Phe | — |
| | Hexyl | | | |
| P1.3.128 | CH2—CH2—CH2— | Ile | Ile | −1.276 |
| | Hexyl | | | |
| P1.3.129 | CH2—CH2—CH2— | Ile | Tyr | −1.767 |
| | Hexyl | | | |
| P1.3.130 | CH2—CH2—CH2— | Ile | Val | −2.425 |
| | Hexyl | | | |
| P1.3.131 | CH2—CH2—CH2— | Thr | Gly | −2.286 |
| | Hexyl | | | |
| P1.3.132 | CH2—CH2—CH2— | Thr | Phe | — |
| | Hexyl | | | |
| P1.3.133 | CH2—CH2—CH2— | Thr | Ile | −1.466 |
| | Hexyl | | | |
| P1.3.134 | CH2—CH2—CH2— | Thr | Tyr | — |
| | Hexyl | | | |
| P1.3.135 | CH2—CH2—CH2— | Thr | Val | 1.545 |
| | Hexyl | | | |
| P1.3.136 | CH2—CH2—CH2— | Lys | Gly | −2.183 |
| | Hexyl | | | |
| P1.3.137 | CH2—CH2—CH2— | Lys | Phe | −5.956 |
| | Hexyl | | | |
| P1.3.138 | CH2—CH2—CH2— | Lys | Ile | −4.148 |
| | Hexyl | | | |
| P1.3.139 | CH2—CH2—CH2— | Lys | Tyr | — |
| | Hexyl | | | |
| P1.3.140 | CH2—CH2—CH2— | Lys | Val | −1.115 |
| | Hexyl | | | |

Other analogues or derivatives of P1.3, include an Fmoc-P1.3 as disclosed below:

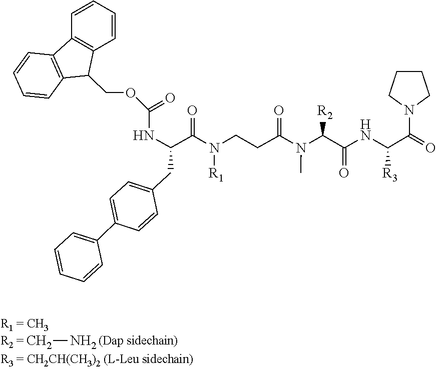

Additional analogues or derivatives of P1.3 include the following:
Wherein R1 is CH₃, R2 is (CH₂)₄NH₂(Lys), R3 is CH(CH₃)₂(Val) and R5 is NCH₃.
And,

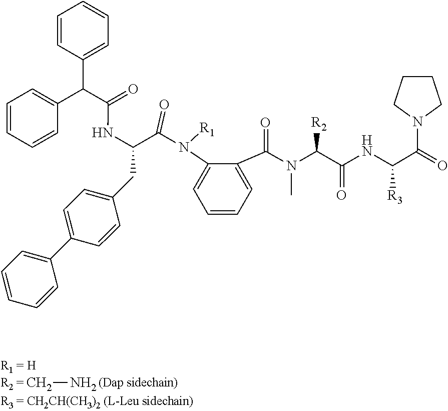

Another analogue or derivative of P1.3 has the following structure;

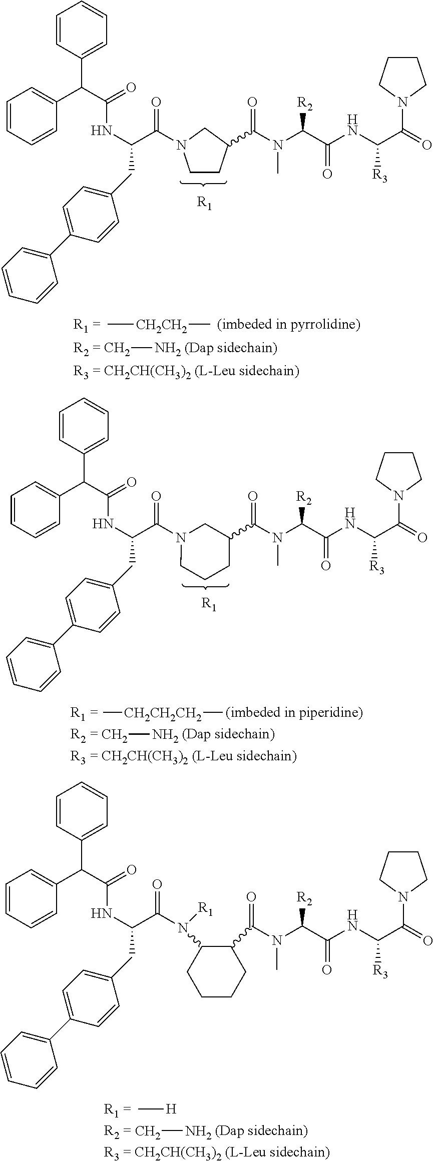

As a general proposition, a therapeutically effective amount or prophylactically effective amount of a therapeutic compound will be administered in a range from about 1 ng/kg body weight/day to about 100 mg/kg body weight/day whether by one or more administrations. In a particular embodiment, therapeutic compound is administered in the range of from about 1 ng/kg body weight/day to about 10 mg/kg body weight/day, about 1 ng/kg body weight/day to about 1 mg/kg body weight/day, about 1 ng/kg body weight/day to about 100 g/kg body weight/day, about 1 ng/kg body weight/day to about 10 g/kg body weight/day, about 1 ng/kg body weight/day to about 1 g/kg body weight/day, about 1 ng/kg body weight/day to about 100 ng/kg body weight/day, about 1 ng/kg body weight/day to about 10 ng/kg body weight/day, about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 g/kg body weight/day, about 10 ng/kg body weight/day to about 10 mg/kg body weight/day, about 10 ng/kg body weight/day to about 1 mg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 1 mg/kg body weight/day to about 100 mg/kg body weight/day, about 1 mg/kg body weight/day to about 10 mg/kg body weight/day, about 1 mg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day, about 10 mg/kg body weight/day to about 10 mg/kg body weight/day, about 10 mg/kg body weight/day to about 1 mg/kg body weight/day, about 10 mg/kg body weight/day to about 100 mg/kg body weight/day, about 100 mg/kg body weight/day to about 100 mg/kg body weight/day, about 100 mg/kg body weight/day to about 10 mg/kg body weight/day, about 100 mg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In other embodiments, a t therapeutic compound is administered at a dose of 500 g to 20 g every three days, or 25 mg/kg body weight every three days.

In other embodiments, a therapeutic compound is administered in the range of about 10 ng to about 100 ng per individual administration, about 10 ng to about 1 g per individual administration, about 10 ng to about 10 g per individual administration, about 10 ng to about 100 mg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 mg per individual administration, about 100 ng to about 10 mg per individual administration, about 100 ng to about 100 mg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 mg to about 10 mg per individual administration, about 1 mg to about 100 mg per individual administration, about 1 mg to about 1 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1 mg per individual administration, about 10 mg to about 10 mg per individual administration, about 10 mg to about 100 mg per individual administration, about 10 mg to about 1000 mg per injection, about 10 mg to about 10,000 mg per individual administration, about 100 mg to about 1 mg per individual administration, about 100 mg to about 10 mg per individual administration, about 100 mg to about 100 mg per individual administration, about 100 mg to about 1000 mg per injection, about 100 mg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The trispecific antibody may be administered daily, every 2, 3, 4, 5, 6 or 7 days, or every 1, 2, 3 or 4 weeks.

In other particular embodiments, the amount of a therapeutic compound may be administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day. As expected, the dosage will be dependent on the condition, size, age and condition of the patient.

Dosages can be tested in several art-accepted animal models suitable for any particular cell proliferative disorder.

In other aspects of this embodiment, a pharmaceutical composition disclosed herein reduces the size of a tumor by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%.

In other aspects of this embodiment, a pharmaceutical composition disclosed herein reduces the size of a tumor by, e.g., about 10%, about 15%, about 20%, about 25%, about 30%, about 35%, about 40%, about 45%, about 50%, about 55%, about 60%, about 65%, about 70%, about 75%, about 80%, about 85%, about 90% or about 95%.

In other aspects of this embodiment, a pharmaceutical composition disclosed herein reduces the size of a tumor by, e.g., no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90% or no more than 95%.

In yet other aspects of this embodiment, a pharmaceutical composition disclosed herein reduces the size of a tumor from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A pharmaceutical composition disclosed herein is in an amount sufficient to allow customary administration to an individual. In aspects of this embodiment, a pharmaceutical composition disclosed herein may be, e.g., at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a pharmaceutical composition.

In other aspects of this embodiment, a pharmaceutical composition disclosed herein may be, e.g., at least 5 mg, at least 10 mg, at least 20 mg, at least 25 mg, at least 50 mg, at least 75 mg, at least 100 mg, at least 200 mg, at least 300 mg, at least 400 mg, at least 500 mg, at least 600 mg, at least 700 mg, at least 800 mg, at least 900 mg, at least 1,000 mg, at least 1,100 mg, at least 1,200 mg, at least 1,300 mg, at least 1,400 mg, or at least 1,500 mg of a pharmaceutical composition. In yet other aspects of this embodiment, a pharmaceutical composition disclosed herein may be in the range of, e.g., about 5 mg to about 100 mg, about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, or about 1,000 mg to about 1,500 mg. In still other aspects of this embodiment, a pharmaceutical composition disclosed herein may be in the range of, e.g., about 10 mg to about 250 mg, about 10 mg to about 500 mg, about 10 mg to about 750 mg, about 10 mg to about 1,000 mg, about 10 mg to about 1,500 mg, about 50 mg to about 250 mg, about 50 mg to about 500 mg, about 50 mg to about 750 mg, about 50 mg to about 1,000 mg, about 50 mg to about 1,500 mg, about 100 mg to about 250 mg, about 100 mg to about 500 mg, about 100 mg to about 750 mg, about 100 mg to about 1,000 mg, about 100 mg to about 1,500 mg, about 200 mg to about 500 mg, about 200 mg to about 750 mg, about 200 mg to about 1,000 mg, about 200 mg to about 1,500 mg, about 5 mg to about 1,500 mg, about 5 mg to about 1,000 mg, or about 5 mg to about 250 mg.

A pharmaceutical composition disclosed herein may comprise a solvent, emulsion or other diluent in an amount sufficient to dissolve a pharmaceutical composition disclosed herein. In other aspects of this embodiment, a pharmaceutical composition disclosed herein may comprise a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a pharmaceutical composition disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

The final concentration of a pharmaceutical composition disclosed herein in a pharmaceutical composition disclosed herein may be of any concentration desired. In an aspect of this embodiment, the final concentration of a pharmaceutical composition in a pharmaceutical composition may be a therapeutically effective amount. In other aspects of this embodiment, the final concentration of a pharmaceutical composition in a pharmaceutical composition may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL or at least 500 mg/mL. In other aspects of this embodiment, the final concentration of a pharmaceutical composition in a pharmaceutical composition may be in a range of, e.g., about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

Aspects of the present specification disclose, in part, treating an individual suffering from cancer. As used herein, the term “treating,” refers to reducing or eliminating in an individual a clinical symptom of cancer; or delaying or preventing in an individual the onset of a clinical symptom of cancer. For example, the term “treating” can mean reducing a symptom of a condition characterized by a cancer, including, but not limited to, tumor size, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with cancer are well known and can be determined by a person of ordinary skill in the art by taking into account factors, including, without limitation, the location of the cancer, the cause of the cancer, the severity of the cancer, and/or the tissue or organ affected by the cancer. Those of skill in the art will know the appropriate symptoms or indicators associated with a specific type of cancer and will know how to determine if an individual is a candidate for treatment as disclosed herein.

In another aspect, a pharmaceutical composition disclosed herein reduces the severity of a symptom of a disorder associated with a cancer. In aspects of this embodiment, a pharmaceutical composition disclosed herein reduces the severity of a symptom of a disorder associated with a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a pharmaceutical composition disclosed herein reduces the severity of a symptom of a disorder associated with a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In aspects of this embodiment, a therapeutically effective amount of a pharmaceutical composition disclosed herein reduces a symptom associated with cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a pharmaceutical composition disclosed herein reduces a symptom associated with cancer by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a pharmaceutical composition disclosed herein reduces a symptom associated with cancer by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In yet other aspects of this embodiment, a therapeutically effective amount of a pharmaceutical composition disclosed herein generally is in the range of about 0.001 mg/kg to about 100 mg/kg and administered, for example, every 3, 5, 7, 10 or 14 days. In aspects of this embodiment, an effective amount of a pharmaceutical composition disclosed herein may be, e.g., at least 0.001 mg/kg, at least 0.01 mg/kg, at least 0.1 mg/kg, at least 1.0 mg/kg, at least 5.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, or at least 50 mg/kg and administered, for example, every 3, 5, 7, 10 or 14 days. In other aspects of this embodiment, an effective amount of a pharmaceutical composition disclosed herein may be in the range of, e.g., about 0.001 mg/kg to about 10 mg/kg, about 0.001 mg/kg/day to about 15 mg/kg, about 0.001 mg/kg to about 20 mg/kg, about 0.001 mg/kg to about 25 mg/kg, about 0.001 mg/kg to about 30 mg/kg, about 0.001 mg/kg to about 35 mg/kg, about 0.001 mg/kg to about 40 mg/kg, about 0.001 mg/kg to about 45 mg/kg, about 0.001 mg/kg to about 50 mg/kg, about 0.001 mg/kg to about 75 mg/kg, or about 0.001 mg/kg to about 100 mg/kg and administered, for example, every 3, 5, 7, 10 or 14 days. In yet other aspects of this embodiment, an effective amount of a pharmaceutical composition disclosed herein may be in the range of, e.g., about 0.01 mg/kg to about 10 mg/kg, about 0.01 mg/kg to about 15 mg/kg, about 0.01 mg/kg to about 20 mg/kg, about 0.01 mg/kg to about 25 mg/kg, about 0.01 mg/kg to about 30 mg/kg, about 0.01 mg/kg to about 35 mg/kg, about 0.01 mg/kg to about 40 mg/kg, about 0.01 mg/kg to about 45 mg/kg, about 0.01 mg/kg to about 50 mg/kg, about 0.01 mg/kg to about 75 mg/kg, or about 0.01 mg/kg to about 100 mg/kg and administered, for example, every 3, 5, 7, 10 or 14 days. In still other aspects of this embodiment, an effective amount of a pharmaceutical composition disclosed herein may be in the range of, e.g., about 0.1 mg/kg to about 10 mg/kg, about 0.1 mg/kg to about 15 mg/kg, about 0.1 mg/kg to about 20 mg/kg, about 0.1 mg/kg to about 25 mg/kg, about 0.1 mg/kg to about 30 mg/kg, about 0.1 mg/kg to about 35 mg/kg, about 0.1 mg/kg to about 40 mg/kg, about 0.1 mg/kg to about 45 mg/kg, about 0.1 mg/kg to about 50 mg/kg, about 0.1 mg/kg to about 75 mg/kg, or about 0.1 mg/kg to about 100 mg/kg and administered, for example, every 3, 5, 7, 10 or 14 days.

In liquid and semi-solid formulations, a concentration of a therapeutic compound disclosed herein typically may be between about 50 mg/mL to about 1,000 mg/mL. In aspects of this embodiment, a therapeutically effective amount of a therapeutic compound disclosed herein may be from, e.g., about 50 mg/mL to about 100 mg/mL, about 50 mg/mL to about 200 mg/mL, about 50 mg/mL to about 300 mg/mL, about 50 mg/mL to about 400 mg/mL, about 50 mg/mL to about 500 mg/mL, about 50 mg/mL to about 600 mg/mL, about 50 mg/mL to about 700 mg/mL, about 50 mg/mL to about 800 mg/mL, about 50 mg/mL to about 900 mg/mL, about 50 mg/mL to about 1,000 mg/mL, about 100 mg/mL to about 200 mg/mL, about 100 mg/mL to about 300 mg/mL, about 100 mg/mL to about 400 mg/mL, about 100 mg/mL to about 500 mg/mL, about 100 mg/mL to about 600 mg/mL, about 100 mg/mL to about 700 mg/mL, about 100 mg/mL to about 800 mg/mL, about 100 mg/mL to about 900 mg/mL, about 100 mg/mL to about 1,000 mg/mL, about 200 mg/mL to about 300 mg/mL, about 200 mg/mL to about 400 mg/mL, about 200 mg/mL to about 500 mg/mL, about 200 mg/mL to about 600 mg/mL, about 200 mg/mL to about 700 mg/mL, about 200 mg/mL to about 800 mg/mL, about 200 mg/mL to about 900 mg/mL, about 200 mg/mL to about 1,000 mg/mL, about 300 mg/mL to about 400 mg/mL, about 300 mg/mL to about 500 mg/mL, about 300 mg/mL to about 600 mg/mL, about 300 mg/mL to about 700 mg/mL, about 300 mg/mL to about 800 mg/mL, about 300 mg/mL to about 900 mg/mL, about 300 mg/mL to about 1,000 mg/mL, about 400 mg/mL to about 500 mg/mL, about 400 mg/mL to about 600 mg/mL, about 400 mg/mL to about 700 mg/mL, about 400 mg/mL to about 800 mg/mL, about 400 mg/mL to about 900 mg/mL, about 400 mg/mL to about 1,000 mg/mL, about 500 mg/mL to about 600 mg/mL, about 500 mg/mL to about 700 mg/mL, about 500 mg/mL to about 800 mg/mL, about 500 mg/mL to about 900 mg/mL, about 500 mg/mL to about 1,000 mg/mL, about 600 mg/mL to about 700 mg/mL, about 600 mg/mL to about 800 mg/mL, about 600 mg/mL to about 900 mg/mL, or about 600 mg/mL to about 1,000 mg/mL.

Dosing can be single dosage or cumulative (serial dosing), and can be readily determined by one skilled in the art. For instance, treatment of a cancer may comprise a one-time administration of an effective dose of a pharmaceutical composition disclosed herein. Alternatively, treatment of a cancer may comprise multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

In one embodiment, a therapeutic compound disclosed herein is capable of reducing the number of cancer cells or tumor size in an individual suffering from a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

In another embodiment, a therapeutic compound disclosed herein is capable of reducing the number of cancer cells or tumor size in an individual suffering from a cancer by, e.g., about 10%, about 15%, about 20%, about 25%, about 30%, about 35%, about 40%, about 45%, about 50%, about 55%, about 60%, about 65%, about 70%, about 75%, about 80%, about 85%, about 90% or about 95% as compared to a patient not receiving the same treatment.

In an embodiment, a therapeutic compound disclosed herein is capable of reducing the number of cancer cells or tumor size in an individual suffering from a cancer by, e.g., no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90% or no more than 95% as compared to a patient not receiving the same treatment. In other aspects of this embodiment, a therapeutic compound is capable of reducing the number of cancer cells or tumor size in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a therapeutic compound and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a therapeutic compound is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a therapeutic compound disclosed herein reduces or maintains a cancer cell population and/or tumor cell size in an individual by, e.g., 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In other aspects of this embodiment, a therapeutically effective amount of a therapeutic compound disclosed herein reduces or maintains a cancer cell population and/or tumor cell size in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%.

In other aspects of this embodiment, a therapeutically effective amount of a therapeutic compound disclosed herein reduces or maintains a cancer cell population and/or tumor cell size in an individual by, e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%.

In yet other aspects of this embodiment, a therapeutically effective amount of a therapeutic compound disclosed herein reduces or maintains a cancer cell population and/or tumor cell size in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A pharmaceutical composition or a therapeutic compound is administered to an individual. An individual is typically a human being, but can be an animal, including, but not limited to, dogs, cats, birds, cattle, horses, sheep, goats, reptiles and other animals, whether domesticated or not. Typically, any individual who is a candidate for treatment is a candidate with some form of cancer, whether the cancer is benign or malignant, a tumor, solid or otherwise, a cancer call not located in a tumor or some other form of cancer. Among the most common types of cancer include, but are not limited to, bladder cancer, breast cancer, colon and rectal cancer, endometrial cancer, kidney cancer, renal cancer, leukemia, lung cancer, melanoma, non-Hodgkins lymphoma, pancreatic cancer, prostate cancer, stomach cancer and thyroid cancer. Pre-operative evaluation typically includes routine history and physical examination in addition to thorough informed consent disclosing all relevant risks and benefits of the procedure.

In an embodiment, a pharmaceutical composition or a therapeutic compound is administered to treat a sarcoma. In an embodiment, a sarcoma is one or more of Angiosarcoma, Chondrosarcoma, Dermatofibrosarcoma protuberans, Desmoplastic small round cell tumors, Epithelioid sarcoma, Ewing sarcoma, Gastrointestinal stromal tumor (GIST), Kaposi's sarcoma, Leiomyosarcoma, Liposarcoma, Malignant peripheral nerve sheath tumors, Myxofibrosarcoma, Osteosarcoma, Rhabdomyosarcoma, Soft tissue sarcoma, Solitary fibrous tumor, Synovial sarcoma and Undifferentiated pleomorphic sarcoma. In another embodiment the sarcoma to be treated is a uterine sarcoma. In a further embodiment, a pharmaceutical composition or a therapeutic compound is administered to treat a uterine cancer. A uterine cancer is either an endometrial cancer or a uterine sarcoma.

In one aspect, a pharmaceutical composition disclosed herein reduces a symptom of a disorder associated with a cancer. In aspects of this embodiment, a pharmaceutical composition disclosed herein reduces a symptom of a disorder associated with a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a pharmaceutical composition disclosed herein reduces a symptom of a disorder associated with a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another aspect, a pharmaceutical composition disclosed herein reduces the frequency of a symptom of a disorder associated with a cancer incurred over a given time period. In aspects of this embodiment, a pharmaceutical composition disclosed herein reduces the frequency of a symptom of a disorder associated with a cancer incurred over a given time period by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a pharmaceutical composition disclosed herein reduces the frequency of a symptom of a disorder associated with a cancer incurred over a given time period by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

The therapeutic method of the present specification may include the step of administering the pharmaceutical composition comprising a therapeutic compound at a pharmaceutically effective amount. The total daily dose should be determined through appropriate medical judgment by a physician and administered once or several times. The specific therapeutically effective dose level for any particular patient may vary depending on various factors well known in the medical art, including the kind and degree of the response to be achieved, pharmaceutical compositions according to whether other agents are used therewith or not, the patient's age, body weight, health condition, gender, and diet, the time and route of administration, the secretion rate of the pharmaceutical composition, the time period of therapy, other drugs used in combination or coincident with the pharmaceutical composition disclosed herein, and like factors well known in the medical arts.

In still another aspect, the present specification provides a use of the therapeutic compound and the pharmaceutical composition including the same in the preparation of drugs for the prevention or treatment of cancer, a neurodegenerative or an infectious disease.

In one embodiment, the dose of the pharmaceutical composition may be administered daily, semi-weekly, weekly, bi-weekly, or monthly. The period of treatment may be for a week, two weeks, a month, two months, four months, six months, eight months, a year, or longer. The initial dose may be larger than a sustaining dose.

In one embodiment, the dose ranges from a weekly dose of at least 0.01 mg/kg, at least 0.25 mg/kg, at least 0.3 mg/kg, at least 0.5 mg/kg, at least 0.75 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 25 mg/kg, or at least 30 mg/kg

In one embodiment, a weekly dose may be at most 1.5 mg/kg, at most 2 mg/kg, at most 2.5 mg/kg, at most 3 mg/kg, at most 4 mg/kg, at most 5 mg/kg, at most 6 mg/kg, at most 7 mg/kg, at most 8 mg/kg, at most 9 mg/kg, at most 10 mg/kg, at most 15 mg/kg, at most 20 mg/kg, at most 25 mg/kg, or at most 30 mg/kg. In a particular aspect, the weekly dose may range from 5 mg/kg to 20 mg/kg. In an alternative aspect, the weekly dose may range from 10 mg/kg to 15 mg/kg.

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, and drug sensitivity, administration route, administration mode, administration frequency, duration of treatment, drugs used in combination or coincident with the composition disclosed herein, and other factors known in medicine.

The pharmaceutical composition comprising the therapeutic compound disclosed herein may further include a pharmaceutically acceptable carrier. For oral administration, the carrier may include, but is not limited to, a binder, a lubricant, a disintegrant, an excipient, a solubilizer, a dispersing agent, a stabilizer, a suspending agent, a colorant, and a flavorant. For injectable preparations, the carrier may include a buffering agent, a preserving agent, an analgesic, a solubilizer, an isotonic agent, and a stabilizer. For preparations for topical administration, the carrier may include a base, an excipient, a lubricant, and a preserving agent.

The disclosed pharmaceutical compositions may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into an ampule as a single dosage form or a multidose container. The pharmaceutical composition may also be formulated into solutions, suspensions, tablets, pills, capsules and long-acting preparations.

On the other hand, examples of the carrier, the excipient, and the diluent suitable for the pharmaceutical formulations include, without limitation, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the pharmaceutical formulations may further include fillers, anti-coagulating agents, lubricants, humectants, flavorants, and antiseptics.

Further, the pharmaceutical composition disclosed herein may have any formulation selected from the group consisting of tablets, pills, powders, granules, capsules, suspensions, liquids for internal use, emulsions, syrups, sterile aqueous solutions, non-aqueous solvents, lyophilized formulations and suppositories.

The pharmaceutical composition may be formulated into a single dosage form suitable for the patient's body, and preferably is formulated into a preparation useful for peptidomimetic drugs according to the typical method in the pharmaceutical field so as to be administered by an oral or parenteral route such as through skin, intravenous, intramuscular, intra-arterial, intramedullary, intramedullary, intraventricular, pulmonary, transdermal, subcutaneous, intraperitoneal, intranasal, intracolonic, topical, sublingual, vaginal, or rectal administration, but is not limited thereto.

The composition may be used by blending with a variety of pharmaceutically acceptable carriers such as physiological saline or organic solvents. In order to increase the stability or absorptivity, carbohydrates such as glucose, sucrose or dextrans, antioxidants such as ascorbic acid or glutathione, chelating agents, low molecular weight proteins or other stabilizers may be used.

The administration dose and frequency of the pharmaceutical composition disclosed herein are determined by the type of active ingredient, together with various factors such as the disease to be treated, administration route, patient's age, gender, and body weight, and disease severity.

The total effective dose of the pharmaceutical compositions disclosed herein may be administered to a patient in a single dose or may be administered for a long period of time in multiple doses according to a fractionated treatment protocol. In the pharmaceutical composition disclosed herein, the content of active ingredient may vary depending on the disease severity.

Preferably, the total daily dose of the peptidomimetic disclosed herein may be approximately 0.0001 μg to 500 mg per 1 kg of body weight of a patient. However, the effective dose of the peptidomimetic is determined considering various factors including patient's age, body weight, health conditions, gender, disease severity, diet, and secretion rate, in addition to administration route and treatment frequency of the pharmaceutical composition. In view of this, those skilled in the art may easily determine an effective dose suitable for the particular use of the pharmaceutical composition disclosed herein. The pharmaceutical composition disclosed herein is not particularly limited to the formulation, and administration route and mode, as long as it shows suitable effects.

Moreover, the pharmaceutical composition may be administered alone or in combination or coincident with other pharmaceutical formulations with or within an active agent showing prophylactic or therapeutic efficacy.

In still another aspect, the present specification provides a method for preventing or treating of cancer, infectious diseases or neurodegenerative diseases comprising the step of administering to a subject a therapeutic compound or a pharmaceutical composition including the same.

Given the teachings and guidance provided herein, those skilled in the art will understand that a formulation described herein can be equally applicable to many types of peptidomimetics and other therapeutic compounds, including those exemplified, as well as others known in the art. Given the teachings and guidance provided herein, those skilled in the art also will understand that the selection of, for example, type(s) or and/or amount(s) of one or more excipients, surfactants and/or optional components can be made based on the chemical and functional compatibility with the biopharmaceutical to be formulated and/or the mode of administration as well as other chemical, functional, physiological and/or medical factors well known in the art. For example, non-reducing sugars exhibit favorable excipient properties when used with polypeptide biopharmaceuticals compared to reducing sugars. Accordingly, exemplary formulations are exemplified further herein with reference to different peptidomimetics. However, the range of applicability, chemical and physical properties, considerations and methodology applied to polypeptide biopharmaceutical can be similarly applicable to biopharmaceuticals other than polypeptide biopharmaceuticals.

In various embodiments, a pharmaceutical composition can include, without limitation, combinations of therapeutic compounds (such as viruses, proteins, antibodies, peptides and the like as described herein) in the pharmaceutical composition. For example, a pharmaceutical composition as described herein can include a single therapeutic compound, such as a peptidometic, for treatment of one or more conditions, including without limitation, a disease. A pharmaceutical composition as described herein also can include, in an embodiment, without limitation, two or more different therapeutic compounds for a single or multiple conditions. Use of multiple therapeutic compounds in a formulation can be directed to, for example, the same or different indications. Similarly, in another embodiment, multiple therapeutic compounds can be used in a formulation to treat, for example, both a pathological condition and one or more side effects caused by the primary treatment. In a further embodiment, multiple therapeutic compounds also can be included, without limitation, in a pharmaceutical composition as described herein to accomplish different medical purposes including, for example, simultaneous treatment and monitoring of the progression of the pathological condition. In an additional embodiment, multiple, concurrent therapies such as those exemplified herein as well as other combinations well known in the art are particularly useful for patient compliance because a single pharmaceutical composition can be sufficient for some or all suggested treatments and/or diagnosis. Those skilled in the art will know those therapeutic compounds that can be admixed for a wide range of combination therapies. Similarly, in various embodiments, a first therapeutic compound can be used with a second or more therapeutic compound and combinations of one or more therapeutic compounds together with one or more other therapeutic compounds, including a small molecule or an antibody pharmaceuticals. Therefore, in various embodiments a formulation is provided containing 1, 2, 3, 4, 5 or 6 or more different therapeutic compounds, as well as, for one or more therapeutic compounds combined with one or more other therapeutic compounds.

In various embodiments, a pharmaceutical composition can include, one or more preservatives and/or additives known in the art. Similarly, a pharmaceutical composition can further be formulated, without limitation, into any of various known delivery formulations. For example, in an embodiment, a pharmaceutical composition can include, surfactants, adjuvant, biodegradable polymers, hydrogels, etc., such optional components, their chemical and functional characteristics are known in the art. Similarly known in the art are pharmaceutical compositions that facilitate rapid, sustained or delayed release of the bioactive agents after administration. A formulation as described can be produced to include these or other formulation components known in the art.

The pharmaceutical composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In various embodiments, the therapeutic compounds in a pharmaceutical composition described herein can, without limitation, be administered to patients throughout an extended time period, such as chronic administration for a chronic condition. The composition can be a solid, a semi-solid or an aerosol and a pharmaceutical compositions is formulated as a tablet, geltab, lozenge, orally dissolved strip, capsule, syrup, oral suspension, emulsion, granule, sprinkle or pellet.

In an embodiment, for oral, rectal, vaginal, parenteral, pulmonary, sublingual and/or intranasal delivery formulations, tablets can be made by compression or molding, optionally with one or more accessory ingredients or additives. In an embodiment, compressed tablets are prepared, for example, by compressing in a suitable tabletting machine, the therapeutic compounds in a free-flowing form such as a powder or granules, optionally mixed with a binder (for example, without limitation, povidone, gelatin, hydroxypropylmethyl cellulose), lubricant, inert diluent, preservative, disintegrant (for example, without limitation, sodium starch glycolate, cross-linked povidone, cross-linked sodium carboxymethyl cellulose) and/or surface-active or dispersing agent.

In an embodiment, molded tablets are made, for example, without limitation, by molding in a suitable tableting machine, a mixture of powdered compounds moistened with an inert liquid diluent. In an embodiment, the tablets may optionally be coated or scored, and may be formulated so as to provide slow or controlled release of the active ingredients, using, for example, without limitation, hydroxypropylmethyl cellulose in varying proportions to provide the desired release profile. In an embodiment, tablets may optionally be provided with a coating, without limitation, such as a thin film, sugar coating, or an enteric coating to provide release in parts of the gut other than the stomach. In an embodiment, processes, equipment, and toll manufacturers for tablet and capsule making are well-known in the art.

In an embodiment, capsule pharmaceutical composition can utilize either hard or soft capsules, including, without limitation, gelatin capsules or vegetarian capsules such as those made out of hydroxymethylpropylcellulose (HMPC). In an embodiment, a type of capsule is a gelatin capsule. In an embodiment, capsules may be filled using a capsule filling machine such as, without limitation, those available from commercial suppliers such as Miranda International or employing capsule manufacturing techniques well-known in the industry, as described in detail in Pharmaceutical Capules, 2.sup.nd Ed., F. Podczeck and B. Jones, 2004. In an embodiment, capsule pharmaceutical composition may be prepared, without limitation, using a toll manufacturing center such as the Chao Center for Industrial Pharmacy & Contract Manufacturing, located at Purdue Research Park.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

Injection devices include pen injectors, auto injectors, safety syringes, injection pumps, infusion pumps, glass prefilled syringes, plastic prefilled syringes and needle free injectors syringes may be prefilled with liquid, or may be dual chambered, for example, for use with lyophilized material. An example of a syringe for such use is the Lyo-Ject™, a dual-chamber pre-filled lyosyringe available from Vetter GmbH, Ravensburg, Germany. Another example is the LyoTip which is a prefilled syringe designed to conveniently deliver lyophilized formulations available from LyoTip, Inc., Camarillo, California, U.S.A. Administration by injection may be, without limitation intravenous, intramuscular, intraperitoneal, or subcutaneous, as appropriate. Administrations by non-injection route may be, without limitation, nasal, oral, cocular, dermal, or pulmonary, as appropriate.

In certain embodiments, kits can comprise, without limitation, one or more single or multi-chambered syringes (e.g., liquid syringes and lyosyringes) for administering one or more pharmaceutical composition described herein. In various embodiments, the kit can comprise pharmaceutical composition for parenteral, subcutaneous, intramuscular or IV administration, sealed in a vial under partial vacuum in a form ready for loading into a syringe and administration to a subject. In this regard, the pharmaceutical composition can be disposed therein under partial vacuum. In all of these embodiments and others, the kits can contain one or more vials in accordance with any of the foregoing, wherein each vial contains a single unit dose for administration to a subject.

The kits can comprise lyophilates, disposed as herein, that upon reconstitution provide pharmaceutical compositions in accordance therewith. In various embodiment the kits can contain a lyophilate and a sterile diluent for reconstituting the lyophilate.

Also described herein, are methods for treating a subject in need of therapy, comprising administering to the subject an effective amount of a pharmaceutical composition as described herein. The therapeutically effective amount or dose of a pharmaceutical composition ormulation will depend on the disease or condition of the subject and actual clinical setting.

In an embodiment, a pharmaceutical composition as described herein can be administered by any suitable route, specifically by parental (including subcutaneous, intramuscular, intravenous and intradermal) administration. It will also be appreciated that the preferred route will vary with the condition and age of the recipient, and the disease being treated. Methods of determining the most effective means and dosage of administration are known to those of skill in the art and will vary, without limitation, with the pharmaceutical composition used for therapy, the purpose of the therapy, and the subject being treated. Single or multiple administrations can be carried out, without limitation, the dose level and pattern being selected by the treating physician. Suitable dosage pharmaceutical compositions and methods of administering the agents are known in the art.

The pharmaceutical compositions as described herein can be used in the manufacture of medicaments and for the treatment of humans and other animals by administration in accordance with conventional procedures.

Also provided herein are combinatorial methods for developing suitable pharmaceutical compositions using combinations of amino acids as an excipient. These methods are effective for developing stable liquid or lyophilized pharmaceutical compositions, and particularly pharmaceutical compositions that comprise one or more therapeutic compounds.

Compositions in accordance with embodiments described herein have desirable properties, such as desirable solubility, viscosity, syringeability and stability. Lyophilates in accordance with embodiments described herein have desirable properties, as well, such as desirable recovery, stability and reconstitution.

In an embodiment, the pH of the pharmaceutical composition is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, or 9.

In an embodiment, the pH of the pharmaceutical composition is from about 3 to about 9, about 4 to about 19, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about 5, about 3 to about 8, about 3 to about 7, about 3 to about 6, about 3 to about 5, about 3 to about 4, about 7 to about 8, about 7 to about 9, about 7 to about 10.

### EXAMPLES

The compositions and methods described herein will be further understood by reference to the following examples, which are intended to be purely exemplary. The compositions and methods described herein are not limited in scope by the exemplified embodiments, which are intended as illustrations of single aspects only. Any methods that are functionally equivalent are within the scope of the invention. Various modifications of the compositions and methods described herein in addition to those expressly described herein will become apparent to those skilled in the art from the foregoing description and accompanying figures. Such modifications fall within the scope of the invention.

### Example 1

Treatment with Compound of Formula (I) Selectively Kills Pancreatic Tumor Cells Expressing Oncogenic KRAS, with a Negligible Effect in Normal Cells

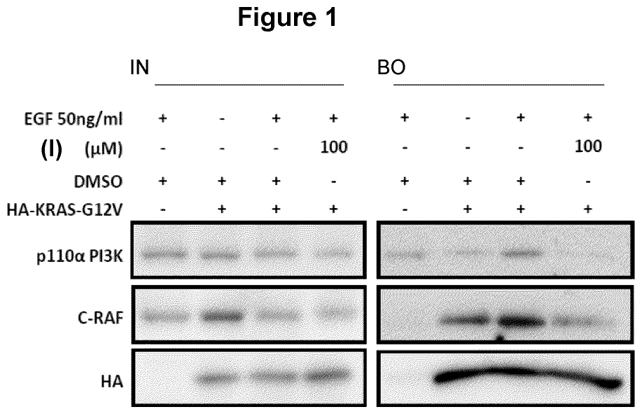

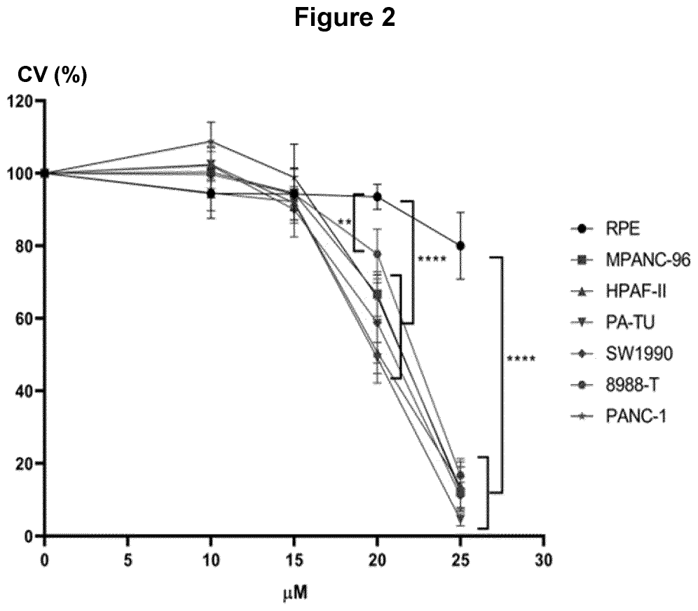

Results shown in Figure. 1 illustrate that a compound of formula (I) reduced PI3K and c-RAF1 interaction with KRAS. Results shown in FIG. 2 illustrate that a compound of formula (I) was able to reduce cell viability with an IC₅₀ of approximately 20 μM in all tumor cell lines, but at these concentrations only affected less than 5% of normal cells. Thus, compound of formula (I) decreases pancreatic tumor cells, but not normal cells.

### General Preparation Process of the Compound of Formula (I) and Analogous Compounds Thereof

Compound of formula (I) and some analogous compounds thereof (the latter not disclosed here) were prepared by means of solid-phase peptide synthesis (SPPS) following an Fmoc/t-Bu strategy. Syntheses were performed on a 100 μmol-scale/each using the 2-chlorotrytil resin as a solid polymeric support. Syntheses were carried out manually in polypropylene syringes fitted with a porous disk at the bottom. While growing the peptidomimetic chain, intermittent manual stirring was carried out to ensure the proper mixing of the reagents. Solvents and soluble reagents were removed by suction. The extent of the amino acid coupling reaction was monitored using either the Kaiser test (primary amines) (cf. E. Kaiser et al.; “Color test for detection of free terminal amino groups in the solid-phase synthesis of peptides”; Anal. Biochem. 1970; vol. 34; pp. 595-598) or the chloranil test (secondary amines) (cf. T. Vojkovsky; “Detection of secondary amines on solid phase”; Pept. Res. 1995; vol. 8; pp. 236-237). In those cases in which the coupling was not fully accomplished, a recoupling step was performed using the standard coupling conditions. Selective N-alkylation of the compound backbone was performed using the method described by S. C. Miller et al. (cf. “Site-selective N-methylation of peptides on solid support”; J. Am. Chem. Soc. 1997; vol. 119; pp. 2301-2302).

The 2-chlorotrityl chloride resin was placed in syringe fitted with a polyethylene porous disk (reaction vessel). The resin was swelled by washes with dichloromethane (DCM) and dimethylformamide (DMF).

Following swelling and preparation of the resin, the protected form of the first Fmoc-protected amino acid of the peptidomimetic to be synthesized was attached to the resin through its carboxylic acid moiety using N,N-Diisopropylethylamine (DIEA) in DMF as coupling agent. To perform the coupling, 0.6 equivalents of the protected amino acid were mixed with few drops of DCM and added to the resin. Later, 5 equivalents of DIEA were added in two times, first 1/3 parts, and 10 min later the remaining 2/3. The reaction was allowed to proceed for 50 min. After that, the not-reacted active points of the polymeric support were capped by pouring methanol (1 mL/g polymeric support) into the reaction mixture. Ten minutes later, the solvents and unreacted reagents were removed by suction. Next, the Fmoc group was removed by treating the resin with 20% piperidine in DMF. The washes were collected and measured by UV spectroscopy to quantify the loading of the first amino acid into the polymeric support.

The subsequent amino acids of the peptidomimetic were coupled using 4 equivalents of Fmoc-protected amino acid, 4 equivalents of 2-(1H-benzotriazole-1-yl)-1,1,3,3-tetramethyluronium tetrafluoborate (TBTU), 8 equivalents of DIEA, and a few drops of DMF which were poured into the reaction vessel containing the polymeric support. This mixture was allowed to react for 75 min. The extent of the coupling reaction was monitored using the Kaiser test or the chloranil test. In case of an incomplete coupling, the reaction was repeated using the same conditions. Washes with DMF (5×1 min) and DCM (5×1 min) were performed during the coupling steps. Once the coupling was completed, the Fmoc group was removed using a mixture of 20% piperidine in DMF (4 mL/g resin, 2×1 min and 1×10 min). The removal of the Fmoc group was monitored using the Kaiser or the chloranil test, which were performed after washing the polymeric support with DMF (5×1 min) and DCM (5×1 min). Subsequent amino acids were coupled using the same reaction conditions until completing the sequence of the target compound.

Amino acid N-alkylation was carried out on-resin. The on-resin process for N-methylation of amino acids used was the following 3 steps methodology (these steps were performed after Fmoc removal of the last coupled amino acid on the peptide sequence anchored onto the polymeric support):
a) Protection and activation of the amino group with o-N-bromosuccinimide (o-NBS).
b) Deprotonation and N-methylation with diazabicyclo[5.4.0]undec-7-ene and dimethylsulfate.
c) o-NBS removal with β-mercaptoethanol and 1,8-Diazabicyclo[5.4.0]undec-7-ene.

Once peptide sequence was completed, the resulting peptide (which still remained anchored onto the polymeric support) was washed with DCM (5×1 min) and dried by suction. Then, the peptide was cleaved from the resin using 5% trifluoroacetic acid (TFA) in DCM. The treatment and DCM washes (5×1 min) were collected and combined to obtain the cleaved peptide from the resin. Then, the collected solvent was evaporated under vacuum until dryness. The crude peptide was diluted with acetonitrile (ACN):H₂O solution (50:50) and lyophilized.

Next, a C-terminal capping was added by diluting the peptide powder in the minimum volume possible of a mixture of DCM, pyrrolidine, 1-hydroxy-7-azabenzotriazole (HOAt) and N-(3-dimethylaminopropyl)-N′-ethylcarbodiimide hydrochloride (EDC-CI). Once the C-terminus capping was completed, the solvent was removed under vacuum, then it was diluted again with a solution of ACN:H₂O 50:50 and lyophilized.

Lateral chains protecting groups were removed by applying a mixture of TFA 95%:triisopropylsilane (TIS) 2.5%: H₂O 2.5% for 1.5 h. All filtrates were pooled and the TFA was evaporated under a N₂ flow stream. Afterwards, the crude was diluted with a mixture of ACN:H₂O (50:50) and lyophilized. Finally, the compound was purified using semipreparative RP-HPLC. Fractions of interest were collected and lyophilized to yield the desired compound.

### Specific Preparation of the Compound of Formula (I)

### Support and Fmoc-Protected Amino Acids:

2-chlorotrityl chloride resin was used as polymeric support. Amino acids used were: Fmoc-Bip-OH, Fmoc-Nme-p-Ala-OH, Fmoc-Dap(Boc)-OH, and Fmoc-Leu-OH.

### Anchoring of First Amino Acid:

2-chlorotrityl chloride resin was initially washed (5×1 min DCM, 5×1 min DMF and 1×5 min DMC) before the addition of the first amino acid. Then, 0.6 equivalents of the amino acid were mixed with few drops of DCM and added to the resin. Later, 5 equivalents of DIEA were added in 2 portions, first, ⅓ part which was allowed to react for 10 min, then the same step was repeated with the remaining ⅔ parts with a reaction time of 50 min. The unreacted active points of the resin were capped using methanol (1 mL/g polymeric support). Afterwards, the reaction mixture was removed by suction and the polymeric support was washed with DMF (5×1 min). Next, the Fmoc group was removed by treating the resin with 20% piperidine in DMF (4 mL/g resin, 2×1 min and 1×10 min). The washes were collected and measured by UV spectroscopy to determine the loading capacity of the first amino acid.

### Peptidomimetic Chain Elongation:

After the coupling of the first amino acid into the polymeric support, the peptidomimetic chain was elongated by pouring a preactivated (3 min) mixture of 4 equivalents of Fmoc-protected amino acid, 4 equivalents of TBTU, 8 equivalents of DIEA, and a few drops of DMF, and poured into the reaction vessel containing the polymeric support. The mixture was allowed to react for 75 min with intermittent manual stirring. After that, reagents and solvents were removed by suction and the polymeric support was washed with DMF (5×1 min) and DCM (5×1 min). The extend of the coupling was monitored by means of the Kaiser test (coupling over a primary amine) or the chloranil test (coupling over a secondary amine). In those cases where the reaction was not completed the coupling step was repeated using the same coupling conditions (4 equivalents of Fmoc-protected amino acid, 4 equivalents of TBTU and 8 equivalents of DIEA and a few drops of DMF, 75 min reaction). After assessing the completeness of the coupling, the Fmoc group was removed with a treatment of 20% piperidine in DMF (4 mL/g resin, 2×1 min and 1×10 min). The removed of the Fmoc group was monitored using the Kaiser or the Chloranil test, which were performed after washing the polymeric support with DMF (5×1 min) and DCM (5×1 min). Subsequent amino acids were coupled using the same reaction conditions until completing the sequence of the target compound.

### Selective N-Methylation of the Diamino Propionic Moiety:

Selective N-methylation was performed as following:
a) Protection and activation of the amino group with o-NBS: 4 equivalents of o-NBS, 3 equivalents of 2,3,5-collidine, and few drops of DMF for 2 times (30 min and 20 min).
b) Deprotonation and N-alkylation: 3 equivalents of 1,8-Diazabicyclo[5.4.0]undec-7-ene in DMF (5 min), and after 5 min 10 equivalents of dimethylsulfate were added on the resin (10 min). This treatment was repeated twice.
c) o-NBS removal: 10 equivalents of β-mercaptoethanol, 5 equivalents of 1,8-Diazabicyclo[5.4.0]undec-7-ene and few drops of DMF for 2 times (10 min and 40 min).
   Cleavage of the Peptide from the Polymeric Support:

The peptide-resin was treated with 5% TFA in DCM (3×15 min, 6 mL). The cleavage mixture and following DCM washes (5×1 min) were collected and combined to obtain the cleaved peptide from the resin. Then, the solvent from the collected mixture was evaporated under vacuum until dryness. Finally, the obtained crude peptide was diluted with ACN/H₂O solution (50:50) and lyophilized. This acidic treatment allowed to keep the protecting side-chain groups of the peptide.

### C-Terminal Capping:

The lyophilizate crude was diluted in the minimum volume possible of DCM, and 3 equivalents of pyrrolidine, 3 equivalents of HOAt, and 3 equivalents of EDC-CI were added. The mixture was allowed to react for 3 h at room temperature under constant stirring. Afterwards, the mixture was washed with saturated solutions of NaHCO₃, NH₄Cl and NaCl (three times each). The organic layer was collected and drayed under vacuum. Afterwards, it was diluted with a mixture of ACN:H₂O (50:50) and lyophilized.

### Removal of the Lateral Chains Protecting Groups:

Lateral chains protecting groups were removed through and acidic mixture treatment, using TFA 95%:TIS 2.5%:H₂O 2.5% (1.5 h). This cleavage mixture was evaporated using a N₂ stream. Afterwards, the crude was diluted with a mixture of ACN:H₂O (50:50) and lyophilized.

### Peptide Purification:

The peptide was purified using semipreparative RP-HPLC. The crude was dissolved in ACN:H₂O (using the lowest amount as possible of ACN:H₂O). The column used was C18 (100 mm×30 mm, 5 μm, 100 Å), using a gradient of 0-100% B in 20 min (A=0.1% TFA in H₂O, B=0.1% TFA in ACN). Flow rate=16 mL/min. Detection=220 nm. Fractions of interest were analyzed by analytical HPLC and HPLC/MS combined and lyophilized.

### Characterization of the Peptide:

The identity of the peptide was confirmed using HPLC-MS. HPLC-MS chromatograms were recorded on a Waters Alliance 2796 separation module system equipped with a Waters 2996 photodiode array detector, quadruple 3100 Mass Detector and a Sunfire C18 column (2.1×100 mm×3.5 μm, 100 Å, Waters), and Masslynx software. Flow rate: 0.3 ml/min, mobile phase: H₂O (0.1% formic acid) and ACN (0.1% formic acid). UV detection: 220 nm. The purity was quantified by analytical HPLC. HPLC chromatograms were recorded on a Waters Alliance 2695 separation module equipped with a 2996 photodiode array detector (PDA) and a Sunfire C18 column (100×4.6 mm×5 μm, 100 Å, Waters), and Empower software. Flow rate: 1.6 mL/min, mobile phase: H₂O (0.1% TFA) and ACN (0.1% TFA). Detection was performed at 220 nm.
Molecular formula: C₄₇H₅₈N₆O₅
Calculated mass: 787.00 g/mol
Mass Identification: [M+H]⁺=787.4 Da
Gradient and retention time (min): gradient of gradient of 0-100% B in 3 minutes (A=0.1% TFA in H2O, B=0.1% TFA in ACN), 3.1 min; gradient of gradient of 40-100% B in 20 minutes (A=0.1% TFA in H₂O, B=0.1% TFA in ACN), 1.9 min.
Purity: 95%.

### Cell Lines and Culture

hTERT-RPE (immortalized retinal pigment epithelial human cells) and HeLa (epithelioid cervix carcinoma cells) both express RAS wild type and were obtained from the American Tissue and Cell Collection (ATCC). MPanc-96, HPAF-II, PA-TU-8902, SW1990, PA-TU 8988T and PANC-1 PDAC cell lines (obtained as described in C. Barcelo et al.: “Ribonucleoprotein HNRNPA2B1 interacts with and regulates oncogenic KRAS in pancreatic ductal adenocarcinoma cells”; Gastroenterology 2014 October; 147(4): 882-892.e8. doi: 10.1053/j.gastro.2014.06.041. Epub 2014 Jul. 3. PMID: 24998203), all express oncogenic mutated KRASG12V.

HeLa and PDAC cells grow in Dulbecco's modified Eagle's medium (DMEM) and hTERT-RPE in DMEM-HAM's F12 (1:1) medium, both supplemented with 10% fetal bovine serum (FBS; Biological Industries, Israel), penicillin, streptomycin, and nonessential amino acids.

### Drug Treatment and EGF-Dependent Signaling and Activation

Cells were seeded in a media containing 10% FBS for 24 h and were serum starved for the next 24 h. Afterwards, they were incubated with different concentrations of the compounds for 2 h. A successive treatment for 10 min with EGF (50 ng/mL; Sigma-Aldrich) was performed in order to activate cell signaling.

### Cell Transfection and Plasmids

HeLa cells were transfected with pEF-HA-KRASG12V plasmid, obtained as described in C. Lopez-Alcala et al. (“Identification of essential interacting elements in K-Ras/calmodulin binding and its role in K-Ras localization.”; J. Biol. Chem. 2008; April 18; 283(16):10621-31. doi: 10.1074/jbc.M706238200. Epub 2008 Jan. 8. PMID: 18182391). Lipofectamine®2000 Transfection Reagent (Invitrogen) was used as a transfection method following the manufacturer's instructions.

### SDS-PAGE, Western Blot and Antibodies

Proteins were resolved by SDS-PAGE and transferred onto PVDF membranes (Immobilon-P, Millipore). Non-specific binding of the antibodies was assessed by incubating the membranes for 1 h at room temperature with a buffer composed of 20 mM Tris-HCl pH 7.5, 150 mM NaCl, 0.05% Tween 20 and 5% bovine serum albumin. Protein expression was determined by probing the blots overnight at 4° C. with the specified antibodies: anti-c-RAF (BD Transduction 610151, 1:500); anti-phospho-c-RAF S338 (Cell Signaling 9427, 1:500); anti-PI3Kp110a (Cell Signaling 4249, 1:1000); anti-AKT (Cell Signaling 9272, 1:1000); anti-phospho-AKT S473 (Cell Signaling 4060, 1:1000); anti-phospho-AKT Thr308 (Cell Signaling 4056, 1:1000); anti-p44/42 MAPK (ERK1/2) (Cell Signaling 9102, 1:2000); anti-phospho-p44/42 MAPK(ERK1/2) T202/Y204 (Cell Signaling 4370, 1:2000); anti-GAP120 (Santa Cruz SC-63, 1:200); anti-HA (Sigma-Aldrich H6908, 1:1000); or anti-α-tubulin (Sigma-Aldrich T9026, 1:2000). Next, after washing the membranes they were incubated with the corresponding HRP-coupled secondary antibodies (goat anti-rabbit BioRad 170-6515 or goat anti-mouse BioRad 170-6516, 1:3000) for 60 min at room temperature and washed again. Protein detection was performed by enhanced chemiluminescence (EZ-ECL, Biological Industries). Emitted light was captioned and quantified (ChemiDoc, BioRad).

For analysis of RAS signaling, cells were lysed in a buffer containing 67 mM Tris-HCl pH 6.8 and 2% SDS, and then the samples were heated at 97° C. for 15 min. After this, the protein concentration of the lysates was assayed using the Lowry method. An aliquot of 15 μg of protein per sample was loaded onto the gels.

### Co-Immunoprecipitation (Co-IP)

Cells were transfected with pEF-HA-KRASG12V plasmid for 24 h and starved for the next 24 h before treatment with the peptides and EGF. Next, an IP with anti-HA antibody crosslinked to agarose beads was performed. Briefly, cells were lysed with a buffer composed of 20 mM Tris-HCl pH 7.5, 100 mM NaCl, 2 mM EDTA, 5 mM MgCl₂, 1% (v/v) Triton X-100, 10% glycerol (v/v), 1 mM dithiothreitol (DTT), plus protease and phosphatase inhibitors (150 nM aprotinin, 20 μM leupeptin, 1 mM phenylmethylsulfonyl, 5 mM sodium fluoride and 0.2 mM sodium orthovanadate) for 10 min on ice. After clarification by centrifugation, the supernatants (500-2000 μg) were incubated with 40-50 μL of anti-HA-tag antibody crosslinked to agarose beads (clone HA-7, Sigma-Aldrich A20956) for 3 h at 4° C. under rotation. Next, the immunocomplexes obtained after 2 min of spinning at 10000 g at 4° C. were washed and subjected to immunoblotting with the corresponding antibodies.

### Cell Viability Assay

10,000 cells in 50 μL of 10% FBS-containing medium, were cultured for 24 h and then treated with the drugs (50 μL final volume) for a further 24 h in each well of a 96-well plate (100 μL final volume). MTS viability assay (CellTiter 96® Aqueous One Solution Cell Proliferation Assay, Promega G3580) was performed following the manufacturer's specifications. The absorbance of each well was measured with a multimode plate reader (Spark, Tecan) at 490 nm. The percentage of cell viability was calculated by dividing the absorbance of each well by the average absorbance of the control wells (which had no significant deviation when Student's t-test was applied).

### Example 2

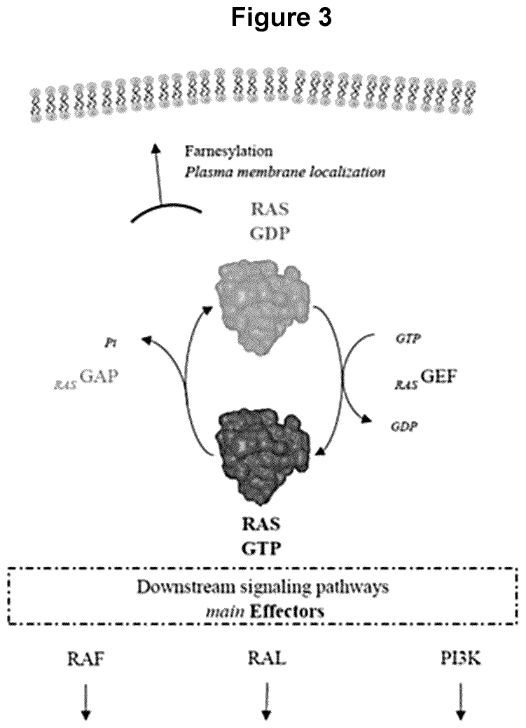

We conducted a preliminary evaluation of the effectors binding sites on the surface of a RAS protein which led us to identify the use of peptidomimetics to modulate the interaction of the RAS protein with those proteins (the effectors following RAS activation) that it interacts at the RAS-effector binding sites (See FIG. 3). The effectors include RAF, RAL and PI3K. This was done using computational approach as described below.

### Computational Hit Identification

Computational approach was applied to afford potent and permeable peptidomimetics as drug candidates. Our approach was to design peptidomimetics that will bind to the RAS-effector binding site, blocking the possibility of RAS to interact with effectors proteins, thus, reducing its oncological activity.

The identification of RAS-effector binding sites was performed by the structural comparison of the GTPase-RAS in complex with several effector proteins (such as phosphoinositide 3-kinase (PI3K), Bry2RBD, RaIGDS, Phospholipase C, NORE1A andRAF) and applying the computational standard protocol.

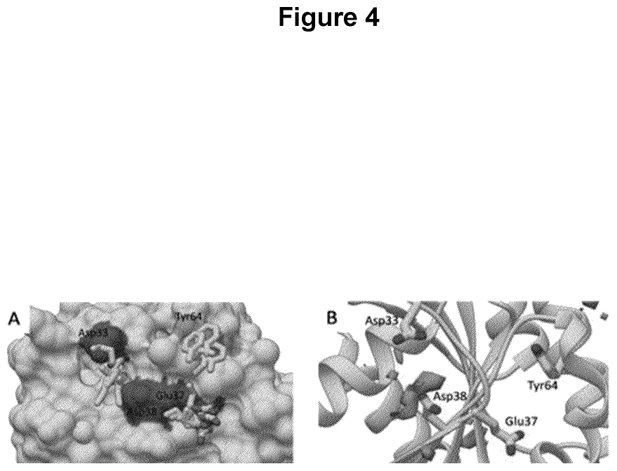

A total of one aromatic and three negatively charged residues (i.e. Asp33, Glu37, Asp38 and Tyr64) were identified on the RAS interface with the RAS effector proteins. We identified several residues on the RAS protein surface, namely Asp33, Glu37, Asp38, and Tyr64, involved in intermolecular contacts with specific residues that are highly conserved among virtually all the effector proteins. We determined that these residues are conserved in the interactions with virtually all the RAS binding sites effector proteins. We also determined that these RAS binding sites are generally highly positive charged between Asp33 and Asp38 (FIG. 4, Table 4) which favors the interaction with a possible binder.

Based on the results obtained from both the analysis of RAS protein crystal structures and MD simulation, the Asp33 and Asp38 residues were selected as the substrate binding site since they were identified by both analyses. These data were subsequently used in virtual screening for putative RAS inhibitors, both to adapt the composition of the peptidomimetic library used as ligands (such as in the hit identification step) as well as to set the position and the size of the docking box (in both the hit identification and hit optimization step).

We created an initial library of more than 80,000 tri- and tetra-peptides, formed by both natural and non-natural amino acids. The tri- and tetra-peptides that were generated contained at least one positive charged residue per peptide. We conducted a peptidomimetic screening using a SMINA docking program. This was accomplished by using the K-RAS GTPase crystal structure as the receptor (PDB 5P21) and setting the binding site around the negatively charged hotspot residues on the surface of the K-RAS GTPase.

### Materials and Methods

### Synthesis of Peptidometics

All compounds were synthesized by means of solid-phase peptide synthesis (SPPS) following an Fmoc/tBu strategy. Syntheses were performed on a 100 μmol-scale/each using the 2-chlorotrityl resin (except for P1.1, for which the H-Rink amide chemmatrix was used). Syntheses were carried out manually in polypropylene syringes fitted with a porous disk at the bottom. While growing the peptide chain intermittent manual stirring was carried out to ensure the proper mixing of the reagents. Solvents and soluble reagents were removed by suction. Amino acid couplings (either L-, D- or non-natural) were performed using 4 equivalents of the Fmoc-protected amino acids, 4 equivalents of 2-(1H-Benzotriazole-1-yl)-1,1,3,3-tetramethylaminium tetrafluoroborate (TBTU) and 8 equivalents of N,N-diisopropylethylamine (DIEA) in dimethylformamide (DMF) (1×75 min). The extent of the reaction was monitored using either the Kaiser test (primary amines) or the Chloranil test (secondary amines). In those cases in which the coupling was not fully accomplished, a recoupling step was performed using the standard coupling conditions. The Fmoc group was removed from the amino acids (once the coupling reaction was successfully completed) using a mixture of 20% of piperidine in DMF (2×1 min and 1×10 min).

Selective N-alkylation of the compound backbone was performed using the method described by Miller et al., which is divided into the following three steps (these steps are performed after Fmoc removal of the amino acid anchored onto the resin that is going to be N-alkylated).

Protection and activation of the amino group with otro-nitrobenzensulfonate (o-NBS): 4 equivalents of o-NBS, 3 equivalents of 2,3,5-Collidine in DMF (1×30 min and 2×20 min), 2) Deprotonation and N-alkylation: 3 equivalents of 1,8-Diazabicyclo[5.4.0]undec-7-ene in DMF are added to the resin (5 min), after that 10 equivalents of desired alkylsulfate are added to the resin(10 min). This treatment is repeated twice. 3) o-NBS removal: two treatments with a mixture of 10 equivalents of β-mercaptoethanol and 5 equivalents of 1,8-Diazabicyclo[5.4.0]undec-7-ene in DMF are performed (1×10 min and 1×40 min).

### Mixed-Solvent Molecular Dynamics (MixMD) Simulation on RAS GTPase Protein

The crystal structure of RAS GTPase protein (PDB code 5P21) was downloaded from the RCSB PDB database (https://www.rcsb.org/) and used as the input structure to perform a 50-ns long explicit mixed-solvent molecular dynamics (MixMD) simulation.

In the first preparatory step, the co-crystallized GppNHp molecule was replaced by the GTP original cofactor, whose parameter files were downloaded from the AMBER parameters database (http://research.bmh.manchester.ac.uk/bryce/amber/). Thus, the overall system was properly protonated and placed in a periodic cubic mixed solvent box composed of benzene, propane, ethanol, propionic acid and ethylamine organic probes properly combined with TIP3Pwater molecules (the minimum distance between protein and edges of the box was set at 10 Å).

The overall protonation, solvation, and parameterization of the system was performed using the Leap module of AmberTool16 and using the ff12 AMBER force field.

As detailed below, a two-step conjugate gradient-based minimization was run followed by a four-step equilibration and a 50-ns long MD simulation using the NAMD simulation package⁵⁷. The SHAKE algorithm and Particle Mesh Ewald (PMD) method were applied (to restrain all bonds to hydrogen atoms and compute long-range Coulomb interactions, respectively) in all simulations. A time step of 2 fs and a cut-off distance for long-range interactions of 12 Å were also set.

Thus, the solvated system was firstly relaxed with a 500-cycle long unrestrained minimization step followed by another 5000 cycles during which harmonic restraints were applied only to the backbone atoms of the protein with a force constant of 5 kcal/(mol Å²). Later, the protein was equilibrated in a four-step protocol in which the system was gradually heated from 0 to 300 K using the Langevin dynamics model and the initial position restraints were regularly relaxed. Thus, 100-ps long MD simulation was performed using NVT conditions (i.e., constant number of molecules (N), Volume (V) and Temperature (T)), restraining backbone atoms with harmonic potential of 20 kcal/(mol Å²) and raising the temperature from 100 to 300K. Then, a 120-ps long heating stage from 300 K to 600 K was run using NVT conditions and restraining backbone atoms with harmonic potential of 10 kcal/(mol Å²). Then the system was cooled for 120 ps from 600 K to 300 K using NVT and restraining backbone atoms with harmonic potential of 10 kcal/(mol Å²). Finally, a 100-ps long simulation at 300 K was performed using NPT conditions (i.e., constant number of molecules (N), Pressure (P) and Temperature (T)) and restraining backbone atoms with harmonic potential of 5 kcal/(mol Å²)

After the equilibration, a 50-ns long simulation at 300 K in NVT conditions was run using minor harmonic restraints of 0.5 kcal/(mol Å²) applied only to backbone atoms.

Finally, the trajectory was used to identify protein surface regions that have a high propensity for ligand binding on the basis of the distribution of organic probes during the trajectory, since the frequency of probe occupation in a given area should be proportional to their binding affinity to this specific area. The positions along the last 25 ns of the simulation of each organic probe is thus integrated into a probe-occupancy map, using the cpptraj module of AmberTools16, and finally visualized as a contour surface corresponding to the region most frequently sampled by each organic probe.

### Virtual Screening of Putative RAS Inhibitors

Using an extended computational survey of the RAS interface, we were able to generate a set of peptidomimetics that could bind to the target binding site with a theoretical potency larger than -8.0 kcal/mol (μM-nM expected experimental inhibitory potency range scale), while exhibiting a good in silico permeability profile, whose average Polar Accessible Surface Area (PASA) was lower than 150 Å². These parameters are representative of the potential activity and permeability of the designed peptidomimetics

To reject the potential of identifying docking false negatives, we conducted short implicit peptide-binding site MD simulations to identify peptidomimetics that exhibited a stable binding mode (RMSD simulation smaller than 3 Å). This method enriches for the selection of compounds that are able to bind to a KRAS-GTPase based on the thermodynamic point, while preserving the binding target area in silico properties and peptides structure.

Two separate and subsequent in silico molecular docking experiments were applied in the screening of two datasets of putative RAS protein inhibitors with the aim of identifying and finally optimizing the hit compound.

In the first molecular docking experiment (hit identification step), a dataset of tri- and tetra-peptidomimetics was built using natural and non-natural amino acids. The N- and C-terminal parts of the compounds were enriched by different capping moieties with different sizes and polarity profiles (such as diphenylactic acid, 2-(4-tert-butylphenoxy)acetic acid, phenylacetic acid, 9-anthracenic acid or benzoic acid for the N-terminal part; and carboxamide, pyrrolidine, pyridine or 3-azopiro[5.5]undecane for the C-terminal part). Based on the results of substrate-binding site analysis, only positively monocharged compounds were selected and thus a library of 80,000 compounds was generated.

### Molecular Docking

The same protocol was used to perform the molecular docking experiments in both the hit identification and optimization step. The three-dimensional structures of all the putative RAS protein inhibitors were created from scratch starting from the primary sequences, parameterized using the AmberTool16 Leap module and ff12 AMBER force field, and finally minimized with the NAMD simulation package. Parameter libraries of non-natural peptidic building blocks (if any) and capping residues were written with the AmberTool16 modules Antechamber and Leap.

The 1.35 Å resolution crystal structure of the RAS GTPase protein structure (PDB code 5P21) was used as the receptor to dock all the putative RAS inhibitors. In the first preparatory step, the co-crystallized GppNHp and water molecules were removed. Thus, hydrogen atoms were added and the entire system was properly protonated using the H++ web server (http://biophysics.cs.vt.edu/H++) with default parameters.

All the docking calculations was performed using SMINA, an energy minimization optimized fork of the AutoDock Vina docking program⁶¹. All the ligands and receptor structures were converted into input files suitable for SMINA using prepare_ligand4.py and prepare_receptor4.py scripts provided by AutoDock Tools. An almost cubic grid box of 60×60×60 size with a grid space of 0.375 Å was adjusted in the RAS effectors binding region and centred around the Asp33 and Asp38 residues. Exhaustiveness, number of modes and energy range were set to 32, 100 and 50 respectively.

Once all the docking simulations had finished, the phi/psi dihedral angle Ramachandran distribution of all the building blocks, the geometry of the corresponding peptide bonds and the intermolecular contacts were calculated for the 20 top ranked docking poses. Therefore, the docking conformations incompatible with phi/psi dihedral angle Ramachandran distribution, bearing no-planar or cis peptide bonds or having any intramolecular contacts, were filtered out. Thus, the top ranked docking poses of each compound (if any) were merged together and sorted according to SMINA docking energy, filtering out all the compounds having a docking energy lower than −8 kcal/mol. Then, the binding stability as well as the predicted cell membrane permeability filters (see below for details) were sequentially applied and after that, the most promising compounds were selected and underwent visual inspection.

For the final selection of peptide candidates, additional factors were taken into account (e.g., peptide binding mode consensus with the predicted substrate binding site location and chemical properties, lack of close orientation of positively charged residues to hydrogen bond donors, close orientation of negatively charged residues to hydrogen bond acceptors and insertion of polar residues into highly hydrophobic clefts).

### RAS Binding Stability Assessment

The docking model of each compound in complex with RAS protein underwent a conjugate gradient minimization, equilibration and 3-ns long implicit solvent MD simulation, using the NAMD simulation package. Thus, the first preparatory step, the overall protonation and parameterization of the system, was performed using the Leap module of AmberTool16 and ff12 AMBER force field. Then, the system was relaxed with a 1000-cycle long minimization, applying harmonic restraints to all backbone atoms of the system (both receptor and ligand) with a force constant of 5 kcal/(mol Å²). Then, a 200-ps long equilibration step was performed by gradually heating the system to 300 K and applying harmonic restraints to the backbone atoms of the protein and the ligand with a force constant of 5 and 2 kcal/(mol Å²), respectively. Finally, a 3-ns long MD simulation was performed applying harmonic restraints only to the backbone atoms of the receptor protein with a force constant of 2 kcal/(mol Å²). In all the simulations, SHAKE⁵⁸ was applied to restrain all bonds to hydrogen atoms while a 2-fs simulation time step and a 12-Å cutoff distance for long-range interaction were set.

Finally, a binding stability assessment was performed to compute the average root-mean-square deviation (LigRMSD_{avg}) of each compound along the last 1.5 ns of the trajectory using the MolSoft ICM Browser (www.molsoft.com)⁶³. Compounds with LigRMSDₐvg values lower than 3 Å were predicted as stable binders and thus selected for the cell membrane permeability evaluation.

### In Silico Permeability Prediction

In the first preparatory step, the three-dimensional structure of each compound was created from scratch and parameterized using the AmberTool16 Leap module and ff12 AMBER force field.

For each compound, a set of 25 2-ns long implicit chloroform-solvated unrestrained molecular dynamics (MD) simulation was performed using the ff12 AMBER force field and NAMD simulation package. Each of the 3D-structures previously generated was firstly used to obtain a small conformational ensemble comprising a total of 25 conformers. Thus, each compound was relaxed by a short 1000-step unrestrained energy minimization and then underwent a short MD simulation conducted for 100 ps, setting the system temperature to 300 K. Finally, 25 conformers were obtained by extracting one trajectory snapshot every 4 ps and used as input for the final chloroform-solvated MD simulation.

Thus, in each simulation the system was firstly energy minimized by 5000 conjugate gradient steps. Then, the system underwent an equilibration process divided into four steps with gradual heating from 0 to 300 K for 100 ps, applying harmonic restraints with a force constant of 0.5 kcal/(mol Å²) on all the heavy atoms in order to preserve initial molecule geometry during heating. During the equilibration, the integration time step was set to 2 fs and the nonbonding cut-off distance to 12 Å. Finally, a production step was run, consisting of an unrestrained MD simulation conducted for 2 ns and setting the system temperature to 300 K. For each compound, all the 25 2-ns long MD trajectories were combined together and a total of 12,500 MD frames were extracted using the cpptraj module of AmberTools16.

Finally, for each compound the average exposure of polar atoms to the solvent during the overall simulation was extracted in terms of average polar accessible solvent area value (polASA_{avg}) on the overall MD frames using the MolSoft ICM Browser. Peptides with polASA_{avg} values lower than 150 Å² were predicted to be permeable by passive diffusion and thus selected for the visual inspection.

### Cell Lines and Culture

hTERT-RPE (immortalized retinal pigment epithelial human cells) expressing RAS wild type were obtained from the American Tissue and Cell Collection (ATCC).

hTERT-RPE in DMEM-HAM's F12 (1:1) medium, both supplemented with 10% fetal bovine serum (Biological Industries, Israel), penicillin, streptomycin, and nonessential amino acids.

### Drug Treatment and EGF-Dependent Signaling Activation

Cells were seeded in a media containing 10% FBS for 24 hours and were serum starved (0.5%) for the next 24 hours. Afterwards, they were incubated with different concentrations of the compounds for 2 hours. A successive treatment for 10 minutes with EGF (50 ng/mL) (Sigma-Aldrich) was performed in order to activate cell signaling.

### SDS-PAGE, Western Blot and Antibodies

Proteins were resolved by SDS-PAGE and transferred onto PVDF membranes (Immobilon-P, Millipore). Non-specific binding of the antibodies was assessed by incubating the membranes for 1 hour at room temperature with a buffer composed of 20 mM Tris-HCl pH 7.5, 150 mM NaCl, 0.05% Tween 20 and 5% bovine serum albumin. Protein expression was determined by probing the blots overnight at 4° C. with the specified antibodies:anti-c-RAF (BD Transduction 610151, 1:500); anti-phospho-c-RAF S338 (Cell Signaling 9427, 1:500); anti-PI3Kp110a (Cell Signaling 4249, 1:1000); anti-AKT (Cell Signaling 9272, 1:1000); anti-phospho-AKT S473 (Cell Signaling 4060, 1:1000); anti-phospho-AKT Thr308 (Cell Signaling 4056, 1:1000); anti-p44/42 MAPK (ERK1/2) (Cell Signaling 9102, 1:2000); anti-phospho-p44/42 MAPK(ERK1/2) T202/Y204 (Cell Signaling 4370, 1:2000); anti-GAP120 (Santa Cruz SC-63, 1:200); anti-HA (Sigma-Aldrich H6908, 1:1000); or anti-α-tubulin (Sigma-Aldrich T9026, 1:2000). Next, after washing the membranes they were incubated with the corresponding HRP-coupled secondary antibodies (goat anti-rabbit BioRad 170-6515 or goat anti-mouse BioRad 170-6516, 1:3000) for 60 minutes at room temperature and washed again. Protein detection was performed by enhanced chemiluminescence (EZ-ECL, Biological Industries). Emitted light was captioned and quantified (ChemiDoc, BioRad).

For analysis of RAS signalling, cells were lysed in a buffer containing 67 mM Tris-HCl pH 6.8 and 2% SDS and then the samples were heated at 97° C. for 15 minutes. After this, the protein concentration of the lysates was assayed using the Lowry method. An aliquot of 15 μg of protein per sample was loaded onto the gels.

### Analysis of Peptidomimetics

After a final visual inspection, nine peptidomimetic sequences were selected for synthesis as set forth in Table 2. The nine sequences includes tri- and tetra-peptidomimetics. All of these sequences have a secondary amine capping at the C-terminal and a hydrophobic group capping at the N-terminal. Permeability threshold was set at 170 Å, with all peptidomimetics that were below this threshold predicted to be permeable.

TABLE 2

| | | | Docking | |
|---|---|---|---|---|
| Compound | | | Score | Permeability |
| Code | Formula | Structure | (Kcal/mol) | (Å²) |
| IP-14-01 | C₄₃H₅₁N₆O₅ | 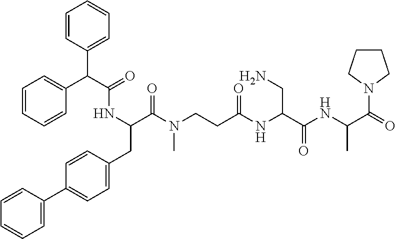 [SMILES: CC(NC(=O)C(CN)NC(=O)CCN(C)C(=O)C(Cc1ccc(-c2ccccc2)cc1)NC(=O)C(c1ccccc1)c1ccccc1)C(=O)N1CCCC1] | −10,2 | 128 |
| IP-14-02 | C₄₂H₅₂N₅O₄ | 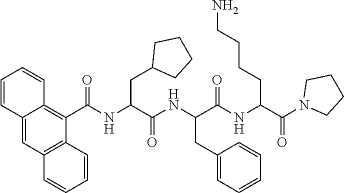 [SMILES: NCCCCC(NC(=O)C(Cc1ccccc1)NC(=O)C(CC1CCCC1)NC(=O)c1c2ccccc2cc2ccccc12)C(=O)N1CCCC1] | −9,7 | 93 |
| IP-14-03 | C₄₂H₆₁N₆O₅ | 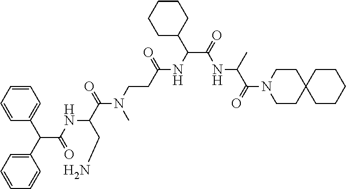 [SMILES: CC(NC(=O)C(NC(=O)CCN(C)C(=O)C(CN)NC(=O)C(c1ccccc1)c1ccccc1)C1CCCCC1)C(=O)N1CCC2(CCCCC2)CC1] | −9,6 | 0105 |
| IP-14-04 | C₅₄H₆₉N₆O₅ | 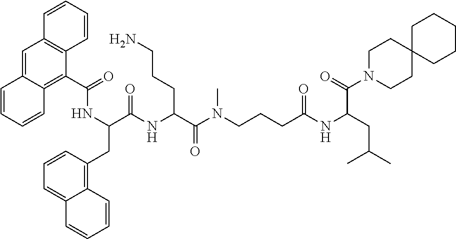 [SMILES: CC(C)CC(NC(=O)CCCN(C)C(=O)C(CCCN)NC(=O)C(Cc1cccc2ccccc12)NC(=O)c1c2ccccc2cc2ccccc12)C(=O)N1CCC2(CCCCC2)CC1] | −9,1 | 123 |
| IP-14-05 | C₄₂H₆₀N₅O₅ | 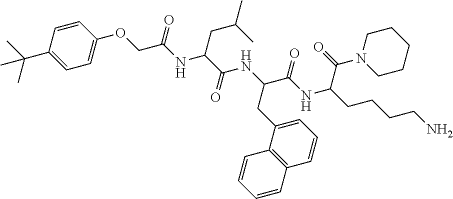 [SMILES: CC(C)CC(NC(=O)COc1ccc(C(C)(C)C)cc1)C(=O)NC(Cc1cccc2ccccc12)C(=O)NC(CCCCN)C(=O)N1CCCCC1] | −8,9 | 111 |
| IP-14-06 | C₄₉H₇₂N₇O₅ | 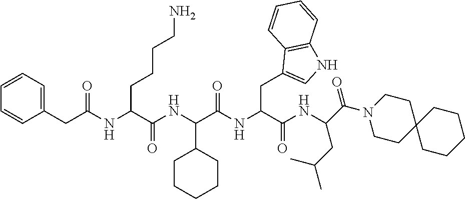 [SMILES: CC(C)CC(NC(=O)C(Cc1c[nH]c2ccccc12)NC(=O)C(NC(=O)C(CCCCN)NC(=O)Cc1ccccc1)C1CCCCC1)C(=O)N1CCC2(CCCCC2)CC1] | −8,4 | 116 |
| IP-14-07 | C₄₆H₆₄N₅O₄ | 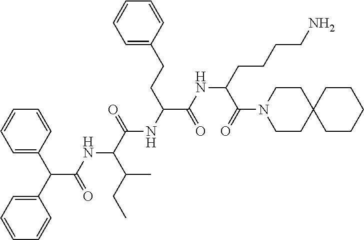 [SMILES: CCC(C)C(NC(=O)C(c1ccccc1)c1ccccc1)C(=O)NC(CCc1ccccc1)C(=O)NC(CCCCN)C(=O)N1CCC2(CCCCC2)CC1] | −8,4 | 86 |
| IP-14-08 | C₄₁H₅₉N₆O₅ | 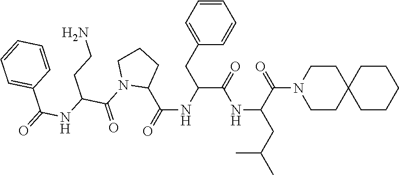 [SMILES: CC(C)CC(NC(=O)C(Cc1ccccc1)NC(=O)C1CCCN1C(=O)C(CCN)NC(=O)c1ccccc1)C(=O)N1CCC2(CCCCC2)CC1] | −8,3 | 133 |
| IP-14-09 | C₄₄H₆₂N₅O₅ | 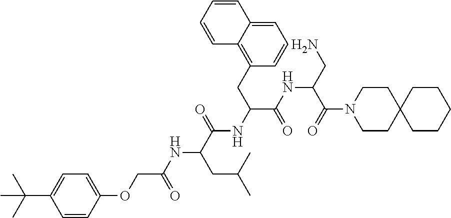 [SMILES: CC(C)CC(NC(=O)COc1ccc(C(C)(C)C)cc1)C(=O)NC(Cc1cccc2ccccc12)C(=O)NC(CN)C(=O)N1CCC2(CCCCC2)CC1] | −8,0 | 104 |

### Experimental Evaluation of Chemical Structures of Table 2

We evaluated the tri- and tetra-peptidomimetics of Table 2 by testing the ability of the peptidomimetic to inhibit the RAS-GTP signaling protein in hTERT-RPE cells. To do this work, we seeded the cells in culture plates for 48 hours. The cells were serum starved (0.5% FCS) for 24 hours. After that period, we added EGF to the cells at a concentration of 50 ng/mL for a period of ten minutes. The EGF was added to the culture to active the RAS signaling pathways. To evaluate the activity of the petidomimetics, 2 hours before the addition of EGF we added one tri- or tetra-peptidomimetic to the culture plates at a concentration of (50 μM) such that each peptidomimetic was tested in a culture plate We ran western blots (WB) to detect the levels of activation of the two Ras signaling pathway (Raf/ERK and PI3K/AKT). GAP120 detection was used as control.

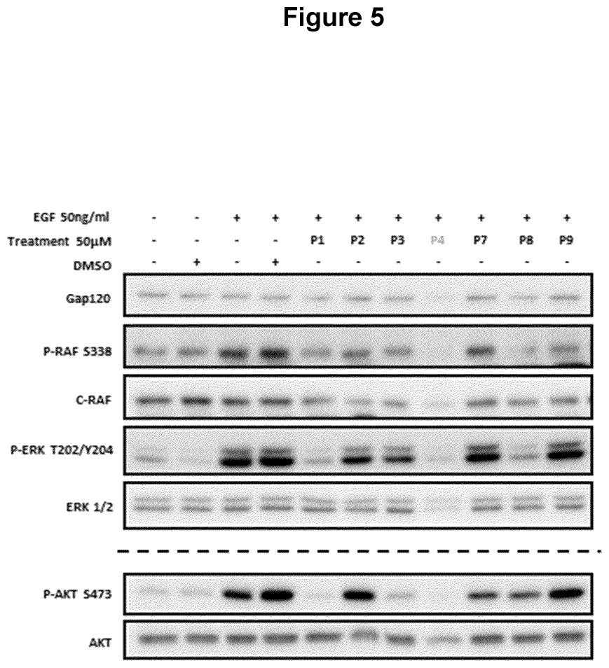

FIG. 5 shows a Western Blot where we evaluated the efficacy of the nine peptidomimetics of Table 2 to inhibit RAS effectors. GTPase activating protein (GAP120) was used as a control.

To consider a peptidomimetic as a positive hit, it should inhibit the activity of the two protein cascades, and as a consequence the phosphorylation of the RAF (P-RAF), AKT (P-AKT) and ERK (P-ERK) should not be observed.

In conducting the analysis, we found that compounds IP-14-05 and IP-14-06 were not soluble when diluted in cell medium. We also found that peptide IP-14-04 formed aggregates and precipitated over the cells causing cell death. As a result, we did not include IP-14-05 and IP-14-06 in the WB (FIG. 5).

As a result, in the end, we only evaluated six of the original nine peptidomimetics in RPE cells. From the six peptidomimetcs we evaluated, we determined that three of them, particularly, IP-14-01, IP-14-03 and IP-14-08 were able to inhibit the two RAS-effectors protein cascades. Of the three, we found that IP-14-01 was the most potent inhibitor when analyzed by WB. (FIG. 5).

As part of our analysis, we evaluated IP-14-01, IP-14-03 and IP-14-08 and their biophysical properties by studying them using a solution of 5% DMSO in water. We conducted a permeability through biological barriers (PAMPA assay) and their internalization in SH-SY5Y cells.

TABLE 3

| Compound | Solubility | | | | |
|---|---|---|---|---|---|
| Code | (mM) | Pe (cm/s) | Transport % | Retention % | Cell internalization % |
| IP-14-01 | 1.333 ± 0.013 | 5.6E−10 ± 3.1E−10 | 0 | 55.7 ± 7.1 | 13.5± |
| IP-14-03 | 0.549 ± 0.090 | 1.6E−09 ± 3.6E−10 | 0 | 77.9 ± 2.4 | 0 |
| IP-14-08 | 1.317 ± 0.022 | 0 | 0 | 97.6 ± 5.0 | 0 |

Table 3 shows the results of the PAMPA assay (Pe, Transport % and Retention %) of IP-14-01, IP-14-03 and IP-14-08, along with the percent of cell internalization to evaluate peptidomimetic permeability. The methods used are well known and would be known to one of skill in the art. As a control, we evaluated the solubility in water which contained 5% DMSO to identify any possible issue related with a low solubility. Data are expressed as the mean±SD.

Solubility was measured in water at 5% DMSO and the three compounds (IP-14-01, IP-14-03 and IP-14-08) showed good solubility, providing assurance that the experiments could be conducted at >100 μM concentrations without the risk of any of the three therapeutic compounds precipitating from the solution. The results of the PAMPA assay showed a high retention %, negligible transport and null permeability Pe for each of the three peptidomimetics. We found that when IP-14-01 was incubated with SH-SY5Y cells for 2 h at 60 μM, 13.5% of the peptideomimetic was taken up by cells. We did not find a similar results for IP-14-03 and IP-14-08 when they were incubated with the same cells.

xxxxx Furthermore, an additional effort was done to reevaluate those peptidomimetics from the first generation that were not soluble in the in vitro assay conditions or formed aggregates as described above when incubated with the RPE cells. For these peptidomimetics, their solubility was studied in PBS with 15% β-cyclodextrin (table 5).

TABLE 4

| Compounds | Conc. in PBS |
|---|---|
| Code | β-cyclodextrin 15% (μM) |
| IP-14-04 | 780 ± 30 |
| IP-14-05 | 849 ± 43 |
| IP-14-06 | 844 ± 4 |
| IP-14-07 | 766 ± 19 |
| IP-14-09 | 949 ± 8 |

Table 4 shows that all these peptidomimetics were diluted and soluble at 1 mM in PBS (phosphate-saline buffer) with 15% β-cyclodextrin. We then put these peptidomimetics under constant agitation for 24 hours. At the end of that time, we centrifuged each of the solutions and then ran the supernatant from each one through an HPLC and compared this result with a 1 mM solution of each compound in ACN/H₂O. This control that was used to determine the real solubility in PBS at 15% β-cyclodextrin. Data are expressed as the mean±SD.

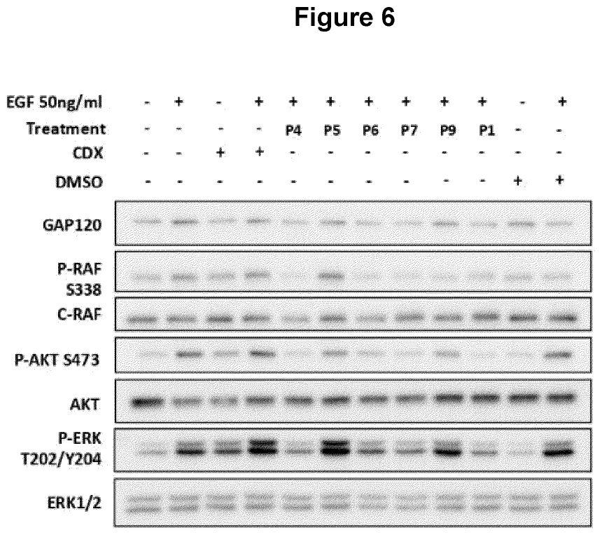

FIG. 6 shows a RAS western blot that we conducted under the same conditions as described above, except in this instance we used β-cyclodextrin at a concentration of 0.5% instead of DMSO to evaluate the compounds IP-14-01 (P1), IP-14-02 (P2) IP-14-03 (P3), IP-14-04 (P4), IP-14-07 (P7), IP-14-08 (P8) and IP-14-09 (P9). We used DMSO to solubilize GTPase activating protein (GAP120) at a concentration of 0.5% diluted in cell medium.

Due to the inhibitory effect of IP-14-01 on the RAS-effector signaling cascades along with its high cellular internalization value, we selected therapeutic compound IP-14-01 for optimization and a second round of computational approach.

### Example 3

Evaluation of Chemical Structures derivatives of IP-14-01

A second generation of peptidomimetics derived from IP-14-01 were designed following the same in silico protocol applied previously. In this regard, we prepared four new peptidomimetics that we eluted once the computational studies were complete.

In the second molecular docking experiment (hit optimization step), a new peptidomimetic dataset was generated starting from P1 primary sequence by the combination of the original building blocks with specific and ad hoc alternative moieties carefully selected in order to increase either the membrane permeability and/or the receptor binding affinity of the original compound.

We designed IPR-471 to increase the number of amino acids on the peptidomimetic backbone. On this basis, the C-terminal was extended by removing the secondary amine that had been used to cap for a proline plus a carboxamide.

IPR-472 had almost the same structure than IP-14-01 but the N-methyl alkylation of the β-Alanine was substituted by a longer carbon chain attached to an aromatic group, propylbenzene. We made this change in order to increase the overall compound hydrophobicity along with an increase of the n-alkyl shielding capacity. We believed a four-carbon chain, instead of a methyl, would provide a certain degree of flexibility that would allow the six-carbon aromatic ring to wrap around the molecule. This would reduce the polarity of IPR-472 in an aqueous environment. We expected that this would increase the number of contacts with the protein surface.

IPR-473 was designed as the most conservative of the IP-14-01 derived peptidomimetics as the only changes were the substitutions of an isoleucine for an alanine and the addition of an N-methylation in one amide bond of the sequence backbone. This new molecule was expected to completely preserve the binding mode of the parent compound but adding a few more contacts in order to slightly optimize its potency.

IPR-474 had two substitutions as compared to IP-14-D1. These were an alanine that was substituted by a cyclohexylglicine and a substitution of an amino acid with a polar group for another amino acid with a polar group. In this case, the new amino acid was a threonine.

Finally, we designed these peptidomimetics to preserve the same sequence ending by keeping the three same amino acids in the same order as well as the diphenyl N-terminal (table 6). These new peptidomimetics are identified in Table 5.

TABLE 5

| | | | Docking |
|---|---|---|---|
| Compound | | | score |
| Code | Formula | Structure | (Kcal/mol) |
| IPR- 471 | C₅₀H₆₆N₆O₆ | 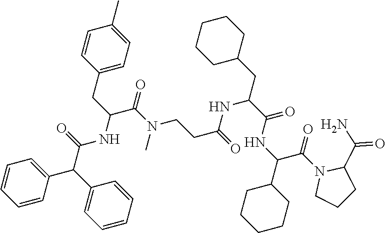 [SMILES: Cc1ccc(CC(NC(=O)C(c2ccccc2)c2ccccc2)C(=O)N(C)CCC(=O)NC(CC2CCCCC2)C(=O)NC(C(=O)N2CCCC2C(N)=O)C2CCCCC2)cc1] | −10,5 |
| IPR- 472 | C₅₁H₅₉N₆O₅ | 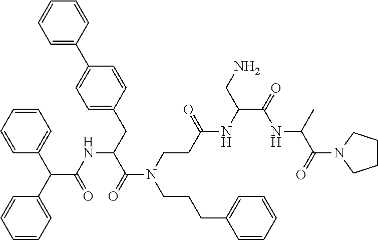 [SMILES: CC(NC(=O)C(CN)NC(=O)CCN(CCCc1ccccc1)C(=O)C(Cc1ccc(-c2ccccc2)cc1)NC(=O)C(c1ccccc1)c1ccccc1)C(=O)N1CCCC1] | −10,2 |
| IPR-473 | C₄₇H₅₉N₆O₅ | 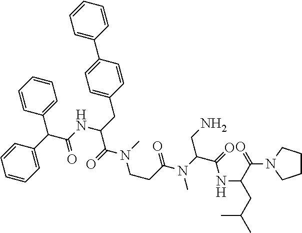 [SMILES: CC(C)CC(NC(=O)C(CN)N(C)C(=O)CCN(C)C(=O)C(Cc1ccc(-c2ccccc2)cc1)NC(=O)C(c1ccccc1)c1ccccc1)C(=O)N1CCCC1] | −9,6 |
| IPR-474 | C₄₉H₅₉N₅O₆ | 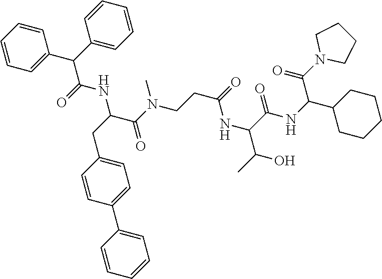 [SMILES: CC(O)C(NC(=O)CCN(C)C(=O)C(Cc1ccc(-c2ccccc2)cc1)NC(=O)C(c1ccccc1)c1ccccc1)C(=O)NC(C(=O)N1CCCC1)C1CCCCC1] | −9,6 |

Table 4 shows that applying the same docking methodology previously used for the screening of the first round of peptidomimetics, a second generation of four new peptidomimetics was generated.

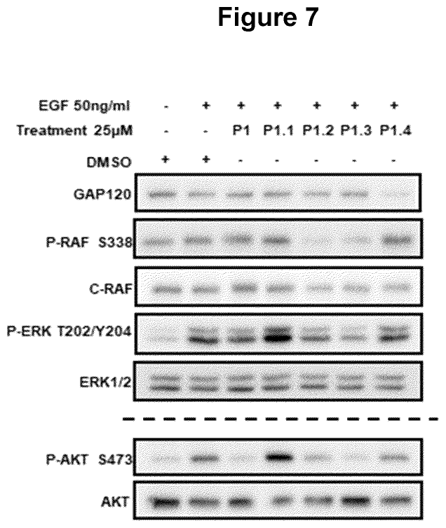

FIG. 7 is a RAS signaling Western Blot from our evaluation of IP-14-01 (P1) and its derived peptidomimetics, IPR-471 (P1.1.), IPR-472(P1.2), IPR-473 (P1.3) and IPR-474 (P1.4). For this experiment, we used the same protocol and conditions as the previously described WE assays disclosed herein (FIG. 5). More particularly, we incubated each of the peptidomimetics compounds in a culture with serum starved hTERT-RPE cells at a concentration of 50 μM dissolved in 0.5% DMSO for two hours and then treated for 10 min with EGF (50 ng/ml). None of these therapeutic compounds exhibited any solubility issues.

As shown in FIG. 7, we found that IPR-471 (P1.1) and IPR-474 (P1.4) were not better than IP-14-01 (P1) from which they were derived. In contrast, we found that IP-14-02 (P1.2) was able to inhibit the RAF and AKT but not ERK. We further determined that IPR-473 (P1.3) was able to inhibit all the two different protein cascades (RAF/ERK and PI3K/AKT) more effectively than even IP-14-01.

Of the peptidomimetic compounds evaluated in FIG. 7, we determined that IPR-473 showed the highest degree of inhibition of the RAS effectors.

We next incubated IP-14-01 at several concentrations with different cancer cell lines and a normal cell line (hTERT-RPE cells). Cell viability was measured by a MTS cell proliferation assay. The same experiment was performed for the parent peptidomimetic IP-14-01. There was not a difference in IP-14-01's ability to kill normal cells versus cancer cells at any incubated concentrations, i.e. the parent peptidomimetic did not show cell-line specificity (data not shown).

We next incubated IPR-473 at several concentrations with different cancer cell lines and a normal cell line (-hTERT-RPE cells). Cell viability was measured by a MTS cell proliferation assay. FIG. 8 shows the results of the MTS viability assay that was used measure the cell viability of seven different cell lines. Cells were placed in a 96-well plate culture containing 10% FBS (Bilogical Industries)-containing medium. (10000 cell per well). These cells were then cultured for 24 hours and then treated with IPR-473 at concentrations of 10 μM, 15 μM, 20 μM and 25 μM for a further 24 hours incubation.

The different cell lines used included human pancreatic cancer cells MPANC-96, human pancreatic adenocarcinoma cells HPAF-II, human pancreatic grade II adenocarcinoma PA-TU, human pancreatic ductaladenocarcinoma SW1990, human pancreas adenocarcinoma 8988-T and human pancreatic ductal carcinoma PANC-1. Each of these comprises a pancreatic tumor cell line. The control as stated was hTERT-RPE cells and that are not cancerigenous.

The results obtained in the cell viability assay as shown in FIG. 8 confirmed the potential therapeutic activity of IPR-473. This therapeutic compound is cytotoxic for cancerigenous cells at concentrations higher than 15 μM. At the same time, it did not had an effect on the hTERT-RPE control cells. So we found that IPR-473 exhibited a high specificity for pancreatic cancer cell lines in a cell proliferation assay.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

1. A compound of formula (I), and pharmaceutically acceptable salts thereof.

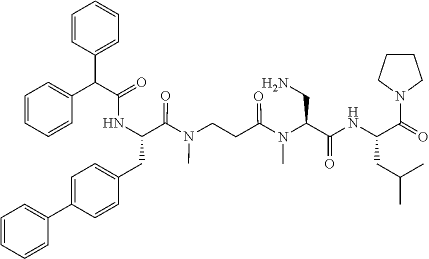 [SMILES: CC(C)C[C@H](NC(=O)[C@H](CN)N(C)C(=O)CCN(C)C(=O)[C@H](Cc1ccc(-c2ccccc2)cc1)NC(=O)C(c1ccccc1)c1ccccc1)C(=O)N1CCCC1]

2: The compound of formula (I) according to claim 1, having the following chemical name: (S)-N-(3-(((S)-3-amino-1-((S)-4-methyl-1-oxo-1-(pyrrolidin-1-yl)pentan-2-ylamino)-1-oxopropan-2-yl)(methyl)amino)-3-oxopropyl)-3-(biphenyl-4-yl)-2-(2,2-diphenylacetamido)-N-methylpropanamide.

3. A pharmaceutical composition comprising a compound of formula (I) or a pharmaceutically acceptable salt thereof, together with a pharmaceutically acceptable excipient, diluent or carrier.

4. A compound of formula (I) or a pharmaceutically acceptable salt thereof, for use in the treatment of human cancer.

5. The compound for use according to claim 4, wherein the human cancer is selected from the group consisting of pancreatic cancer, lung cancer and colorectal cancer.

6. The compound for use according to claim 5, wherein the human cancer is pancreatic cancer.

7. The compound for use according to claim 6, wherein the pancreatic cancer is pancreatic ductal adenocarcinoma, PDAC.
